SEQUENCE LISTING (21 sequences)

SEQ ID NO: 1 - protein, 245 aa, Bacteriophage T4
MKSGIYQIKNTLNNKVYVGSAKDFEKRWKRHFKDLEKGCHSSIKLQRSFNKHGNVFECSILEEIPYEKDLIIERENFWIKELNSKINGYNIADATFGDTCSTHPLKEEIIKKRSETVKAKMLKLGPDGRKALYSKPGSKNGRWNPETHKFCKCGVRIQTSAYTCSKCRNRSGENNSFFNHKHSDITKSKISEKMKGKKPSNIKKISCDGVIFDCAADAARHFKISSGLVTYRVKSDKWNWFYINA

SEQ ID NO: 2 - protein, 168 aa, Bacteriophage T4
MKSGIYQIKNTLNNKVYVGSAKDFEKRWKRHFKDLEKGCHSSIKLQRSFNKHGNVFECSILEEIPYEKDLIIERENFWIKELNSKINGYNIADATFGDTCSTHPLKEEIIKKRSETVKAKMLKLGPDGRKALYSKPGSKNGRWNPETHKFCKCGVRIQTSAYTCSKCR

SEQ ID NO: 3 - DNA, 510 bp, Bacteriophage T4
atgaaaagcggaatttatcagattaaaaatactttaaacaataaagtatatgtaggaagtgctaaagattttgaaaagagatggaagaggcattttaaagatttagaaaaaggatgccattcttctataaaacttcagaggtcttttaacaaacatggtaatgtgtttgaatgttctattttggaagaaattccatatgagaaagatttgattattgaacgagaaaatttttggattaaagagcttaattctaaaattaatggatacaatattgctgatgcaacgtttggtgatacatgttctacgcatccattaaaagaagaaattattaagaaacgttctgaaactgttaaagctaagatgcttaaacttggacctgatggtcggaaagctctttacagtaaacccggaagtaaaaacgggcgttggaatccagaaacccataagttttgtaagtgcggtgttcgcatacaaacttctgcttatacttgtagtaaatgcagaggatcc

SEQ ID NO: 4 - DNA, 37 bp, Bacteriophage T4
ccatggtaccagatctatgaaaagcggaatttatcag

SEQ ID NO: 5 - DNA, 31 bp, Bacteriophage T4
ccggatcctctgcatttactacaagtataag

SEQ ID NO: 6 - DNA, 4858 bp, Artificial sequence
  fusion of I-TevI from Bacteriophage T4 and AvrXa7 from Xanthomonas oryzae
atgaaaagcggaatttatcagattaaaaatactttaaacaataaagtatatgtaggaagtgctaaagattttgaaaagagatggaagaggcattttaaagatttagaaaaaggatgccattcttctataaaacttcagaggtcttttaacaaacatggtaatgtgtttgaatgttctattttggaagaaattccatatgagaaagatttgattattgaacgagaaaatttttggattaaagagcttaattctaaaattaatggatacaatattgctgatgcaacgtttggtgatacatgttctacgcatccattaaaagaagaaattattaagaaacgttctgaaactgttaaagctaagatgcttaaacttggacctgatggtcggaaagctctttacagtaaacccggaagtaaaaacgggcgttggaatccagaaacccataagttttgtaagtgcggtgttcgcatacaaacttctgcttatacttgtagtaaatgcagaggatctgccaccatggatcccattcgttcgcgcacgccaagtcctgcccgcgagcttctgcccggaccccaaccggatagggttcagccgactgcagatcgggggggggctccgcctgctggcggccccctggatggcttgcccgctcggcggacgatgtcccggacccggctgccatctccccctgcgccctcgcctgcgttctcggcgggcagcttcagcgatctgctccgtcagttcgatccgtcgcttcttgatacatcgcttcttgattcgatgcctgccgtcggcacgccgcatacagcggctgccccagcagagtgggatgaggtgcaatcgggtctgcgtgcagccgatgacccgccacccaccgtgcgtgtcgctgtcactgccgcgcggccgccgcgcgccaagccggccccgcgacggcgtgcggcgcaaccctccgacgcttcgccggccgcgcaggtggatctacgcacgctcggctacagtcagcagcagcaagagaagatcaaaccgaaggtgcgttcgacagtggcgcagcaccacgaggcactggtgggccatgggtttacacacgcgcacatcgttgcgctcagccaacacccggcagcgttagggaccgtcgctgtcaagtatcagcacataatcacggcgttgccagaggcgacacacgaagacatcgttggcgtcggcaaacagtggtccggcgcacgcgccctggaggccttgctcacgaaggcgggggagttgagaggtccgccgttacagttggacacaggccaacttctcaagattgcaaaacgtggcggcgtgaccgcagtggaggcagtgcatgcatggcgcaatgcactgacgggtgcccccctgaacctgaccccggaccaagtggtggccatcgccagcaatattggcggcaagcaggcgctggagacggtacagcggctgttgccggtgctgtgccaggaccatggcctgaccccggaccaggtcgtggccatcgccagccatggcggcggcaagcaggcgctggagacggtgcagcggctgttgccggtgctgtgccaggaccatggcctgaccccggaccaggtggtggccatcgccagcaatattggcggcaagcaggcgctagagacggtgcagcggctgttgccggtgctgtgccaggcccatggcctgaccccggaccaggtcgtggccatcgccagcaatattggcggcaagcaggcgctggagacggtgcagcggctgttgccggtgctgtgccaggaccatggcctgaccccggcccaggtggtggccatcgccagcaatagtggcggcaagcaggcgctggagacggtgcagcggctgttgccggtgctgtgccaggaccatggcctgaccccggaccaagtcgtggccatcgccagccacgatggcggcaagcaggcgctggagacgctgcagcggctgttgccggtgctgtgccaggaccatggcctgaccccggaccaggtcgtggccatcgccaacaataacggcggcaagcaggcgctggagacgctgcagcggctgttgccggtgctgtgccaggaccatggcctgaccccggaccaagtggtggccatcgccagccacgatggcggcaagcaggcgctggagacggtgcagcggctgttgccggtgctgtgccaggaccatggcctgaccccggaccaggtggtggccatcgccagccacgatggcggcaagcaggcgctggagacggtgcagcggctgttgccggtgctgtgccaggaccatggcctgaccccggcccaagtggtggccatcgccagccacgatggcggcaagcaggcgctggagacggtgcagcggctgttgccggtgctgtgccaggaccatggcctgaccccggaccaggtggtggccatcgccagcaatagcggcggcaagcaggcgctggagacggtacagcggctgttgccggtgctgtgccaggaccatggactgaccccggaccaggtcgtggccatcgccagcaatggcggcaagcaggcgctggagacggtacagcggctgttgccggtgctgtgccaggaccatggcctgaccccggaccaggtcgtggccatcgccagcaatggcggcaagcaggcgctggagacggtgcagcggctgttgccggtacagcggctgttgccggtgctgtgccaggaccatggcctgacccaggaccaggtggtggccatcgccagccacgatggcggcaagcaggcgctggagacggtgcagcggctgttgccggtgctgtgccaggaccatggcctgaccccggaccaagtggtggccatcgccagccacgatggcggcaaacaggcgctggagacggtgcagcggctgttgccggtgctgtgccaggaccatggcctgaccccggaccaggtggtggccatcgccagcaatagtggcggcaagcaggcgctggagacggtgcagcggctgttgccggtgctgtgccaggaccatggcctgaccccggaccaagtggtggccatcgccagcaatagtggcggcaagcaggcgctggagacggtgcagcggctgttgccggtgctgtgccaggaccatggcctgaccccggaccaggtggtggccatcgccagcaataacggcggcaagcaggcgctggagacggtgcagcggctgttgccggtgctgtgccaggaccatggcctgaccccggaccaggtcgtggccatcgccaacaataacggcggcaagcaggcgctggagacggtgcagcggctgttgccggtgctgtgccaggaccatggcctgaccccggcgcaggtggtggccatcgccagcaatattggcggcaagcaggcgctggagacggtgcagcggctgttgccggtgctgtgccaggaccatggcctgaccctggaccaggtggtggccattgccagcaatggcggcagcaaacaggcgctagagacggtgcagcggctgttgccggtgctgtgccaggaccatggcctgaccccggaccaagtggtggccatcgccaacaataacggcggcaagcaggcgctggagacggtgcagcggctgttgccggtgctgtgccaggaccatggcctgaccccggaccaggtcgtggccatcgccagcaatattggcggcaagcaggcgctggagacggtgcagcggctgttgccggtgctgtgccaggaccatggcctgaccctggaccaggtggtggccatcgccagcaatggcggcaagcaggcgctggagacggtgcagcggctgttgccggtgctgtgccaggaccatggcctgaccccgaaccaggtggtggccatcgccagcaatagtggcggcaagcaggcgctggagacgcctgaccccgaaccaggtggtggccatcgccagcaatggcggcaagcaggcgctggagagcattgttgcccagttatctcgccctgatccggcgttggccgcgttgaccaacgaccacctcgtcgccttggcctgcctcggcggacgtcctgccctggatgcagtgaaaaagggattgccgcacgcgccggaattgatcagaagaatcaatcgccgcattcccgaacgcacgtcccatcgcgttcccgacctcgcgcacgtggttcgcgtgcttggttttttccagagccactcccacccagcgcaagcattcgatgacgccatgacgcagttcgagatgagcaggcacggcttggtacagctctttcgcagagtgggcgtcaccgaattcgaagcccgctacggaacgctccccccagcctcgcagcgttgggaccgtatcctccaggcatcagggatgaaaagggccaaaccgtcccctacttcagctcaaacaccggatcaggcgtctttgcatgcagattacaaggacgacgacgacaagaaggattacaaggacgacgacgacaagaagggtcgacccagcccaatgcacgagggagatcagacgcgggcaagcagccgtaaacggtcccgatcggatcgtgctgtcaccggcccctccacacagcaatctttcgaggtgcgcgttcccgaacagcaagatgcgctgcatttgcccctcagctggagggtaaaacgcccgcgtaccaggatcgggggcggcctcccggatcctggtacgcccatcgctgccgacctggcagcgtccagcaccgtgatgtgggaacaagatgcggcccccttcgcaggggcagcggatgatttcccggcattcaacgaagaggagctcgcatggttgatggagctattgcctcagtcaggctcagtcggagggacgatcaagcttctagactag

SEQ ID NO: 7 - DNA, 27 bp, Xanthomonas oryzae
tataaaccccctccaaccaggtgctaa

SEQ ID NO: 8 - DNA, 4173 bp, Artificial sequence
  Bacteriophage T4 and Xanthomonas u3Ar fusion
atgaaaagcggaatttatcagattaaaaatactttaaacaataaagtatatgtaggaagtgctaaagattttgaaaagagatggaagaggcattttaaagatttagaaaaaggatgccattcttctataaaacttcagaggtcttttaacaaacatggtaatgtgtttgaatgttctattttggaagaaattccatatgagaaagatttgattattgaacgagaaaatttttggattaaagagcttaattctaaaattaatggatacaatattgctgatgcaacgtttggtgatacatgttctacgcatccattaaaagaagaaattattaagaaacgttctgaaactgttaaagctaagatgcttaaacttggacctgatggtcggaaagctctttacagtaaacccggaagtaaaaacgggcgttggaatccagaaacccataagttttgtaagtgcggtgttcgcatacaaacttctgcttatacttgtagtaaatgcagaggatctgccaccatggatcccattcgttcgcgcacgccaagtcctgcccgcgagcttctgcccggaccccaaccggatagggttcagccgactgcagatcgggggggggctccgcctgctggcggccccctggatggcttgcccgctcggcggacgatgtcccggacccggctgccatctccccctgcgccctcgcctgcgttctcggcgggcagcttcagcgatctgctccgtcagttcgatccgtcgcttcttgatacatcgcttcttgattcgatgcctgccgtcggcacgccgcatacagcggctgccccagcagagtgggatgaggtgcaatcgggtctgcgtgcagccgatgacccgccacccaccgtgcgtgtcgctgtcactgccgcgcggccgccgcgcgccaagccggccccgcgacggcgtgcggcgcaaccctccgacgcttcgccggccgcgcaggtggatctacgcacgctcggctacagtcagcagcagcaagagaagatcaaaccgaaggtgcgttcgacagtggcgcagcaccacgaggcactggtgggccatgggtttacacacgcgcacatcgttgcgctcagccaacacccggcagcgttagggaccgtcgctgtcaagtatcagcacataatcacggcgttgccagaggcgacacacgaagacatcgttggcgtcggcaaacagtggtccggcgcacgcgccctggaggccttgctcacgaaggcgggggagttgagaggtccgccgttacagttggacacaggccaacttctcaagattgcaaaacgtggcggcgtgaccgcagtggaggcagtgcatgcatggcgcaatgcactgacgggtgcccccctgaacctgaccccggaccaagtggtggccatcgccagcaatattggcggcaagcaggcgctggagacggtgcagcggctgttgccggtgctgtgccaggaccatggcctgaccccggaccaggtcgtggccatcgccagcaatggcggcggcaagcaggccttggagacggtgcagcggctgttgccggtgctgtgccaggcccatggcctgaccccggcccaagtggtggccatcgccagcaatggcggcggcaagcaggctctggagacggtacagcggctgttgccggtgctgtgccaggaccatggcctgaccccggaccaggtcgtggccatcgccagcaatattggcggcaagcaggcgttggagacggtacagcggctgttgccggtgctgtgccaggaccatggcctgaccccggaccaggtggtggccatcgccagcaatattggcggcaagcaggctttggagacggtacagcggctgttgccggtgctgtgccaggaccatggcctgaccccggaccaggtggtggccatcgccagcaatattggcggcaagcaggcattggagacggtacagcggctgttgccggtgctgtgccaggaccatggcctgaccccggaccaggtcgtggccatcgccagcaatggcggcggcaagcaggccctggagacggtacagcggctgttgccggtgctgtgccaggaccatggcctgaccccggaccaggtggtggccatcgccagcaatattggcggcaagcaggcactggagacggtacagcggctgttgccggtgctgtgccaggaccatggcctgaccccggaccaggtggtggccatcgccaacaataacggcggcaagcaggcactggaaacactgcagcggctgttgccggtgctgtgccaggaccatggcctgacccaggaccaggtggtggccatcgccagccacgatggcggcaagcaggccttggagacggtgcagcggctgttgccggtgctgtgccaggcccatggcctgaccccggcccaagtggtggccatcgccagcaatggcggcggcaagcaggctctggagacggtacagcggctgttgccggtgctgtgccaggaccatggcctgaccccggaccaggtcgtggccatcgccagcaatggcggcggcaagcaggcgttggagacggtacagcggctgttgccggtgctgtgccaggaccatggcctgaccccggaccaggtggtggccatcgccaacaataacggcggcaagcaggctttggagacggtacagcggctgttgccggtgctgtgccaggaccatggcctgaccccggaccaggtcgtggccatcgccaacaataacggcggcaagcaggcattggagacggtacagcggctgttgccggtgctgtgccaggaccatggcctgacccaggaccaggtggtggccatcgccagccacgatggcggcaagcaggccctggagacggtacagcggctgttgccggtgctgtgccaggaccatggcctgaccccggaccaggtggtggccatcgccagcaatattggcggcaagcaggcactggagacggtacagcggctgttgccggtgctgtgccaggaccatggcctgaccccggaccaggtcgtggccatcgccaacaataacggcggcaagcaggcactggagactgtacagcggctgttgccggtgctgtgccaggaccatggcctgacccaggaccaggtggtggccatcgccagccacgatggcggcaagcaggcactggagacggtacagcggctgttgccggtgctgtgccaggaccatggcctgaccccgaaccaggtggtggccatcgccagcaatggcggcaagcaggcgctggagagcattgttgcccagttatctcgccctgatccggcgttggccgcgttgaccaacgaccacctcgtcgccttggcctgcctcggcggacgtcctgccctggatgcagtgaaaaagggattgccgcacgcgccggaattgatcagaagaatcaatcgccgcattcccgaacgcacgtcccatcgcgttcccgacctcgcgcacgtggttcgcgtgcttggttttttccagagccactcccacccagcgcaagcattcgatgacgccatgacgcagttcgagatgagcaggcacggcttggtacagctctttcgcagagtgggcgtcaccgaattcgaagcccgctacggaacgctccccccagcctcgcagcgttgggaccgtatcctccaggcatcagggatgaaaagggccaaaccgtcccctacttcagctcaaacaccggatcaggcgtctttgcatgcagattacaaggacgacgacgacaagaaggattacaaggacgacgacgacaagaagggtcgacccagcccaatgcacgagggagatcagacgcgggcaagcagccgtaaacggtcccgatcggatcgtgctgtcaccggcccctccacacagcaatctttcgaggtgcgcgttcccgaacagcaagatgcgctgcatttgcccctcagctggagggtaaaacgcccgcgtaccaggatcgggggcggcctcccggatcctggtacgcccatcgctgccgacctggcagcgtccagcaccgtgatgtgggaacaagatgcggcccccttcgcaggggcagcggatgatttcccggcattcaacgaagaggagctcgcatggttgatggagctattgcctcagtcaggctcagtcggagggacgatcaagcttctagactag

SEQ ID NO: 9 - DNA, 19 bp, Artificial sequence
  fusion of I-TevI from Bacteriophage T4 and Uv3Ar from Xanthomonas oryzae
tattaaatagcttggcagc

SEQ ID NO: 10 - DNA, 3969 bp, Artificial sequence
  fusion of I-TevI from Bacteriophage T4 and Uv3b from Xanthomonas oryzae
atgaaaagcggaatttatcagattaaaaatactttaaacaataaagtatatgtaggaagtgctaaagattttgaaaagagatggaagaggcattttaaagatttagaaaaaggatgccattcttctataaaacttcagaggtcttttaacaaacatggtaatgtgtttgaatgttctattttggaagaaattccatatgagaaagatttgattattgaacgagaaaatttttggattaaagagcttaattctaaaattaatggatacaatattgctgatgcaacgtttggtgatacatgttctacgcatccattaaaagaagaaattattaagaaacgttctgaaactgttaaagctaagatgcttaaacttggacctgatggtcggaaagctctttacagtaaacccggaagtaaaaacgggcgttggaatccagaaacccataagttttgtaagtgcggtgttcgcatacaaacttctgcttatacttgtagtaaatgcagaggatctgccaccatggatcccattcgttcgcgcacgccaagtcctgcccgcgagcttctgcccggaccccaaccggatagggttcagccgactgcagatcgggggggggctccgcctgctggcggccccctggatggcttgcccgctcggcggacgatgtcccggacccggctgccatctccccctgcgccctcgcctgcgttctcggcgggcagcttcagcgatctgctccgtcagttcgatccgtcgcttcttgatacatcgcttcttgattcgatgcctgccgtcggcacgccgcatacagcggctgccccagcagagtgggatgaggtgcaatcgggtctgcgtgcagccgatgacccgccacccaccgtgcgtgtcgctgtcactgccgcgcggccgccgcgcgccaagccggccccgcgacggcgtgcggcgcaaccctccgacgcttcgccggccgcgcaggtggatctacgcacgctcggctacagtcagcagcagcaagagaagatcaaaccgaaggtgcgttcgacagtggcgcagcaccacgaggcactggtgggccatgggtttacacacgcgcacatcgttgcgctcagccaacacccggcagcgttagggaccgtcgctgtcaagtatcagcacataatcacggcgttgccagaggcgacacacgaagacatcgttggcgtcggcaaacagtggtccggcgcacgcgccctggaggccttgctcacgaaggcgggggagttgagaggtccgccgttacagttggacacaggccaacttctcaagattgcaaaacgtggcggcgtgaccgcagtggaggcagtgcatgcatggcgcaatgcactgacgggtgcccccctgaacctgaccccggaccaagtggtggccatcgccagcaatattggcggcaagcaggcgctggagacggtacagcggctgttgccggtgctgtgccaggaccatggcctgacccaggaacaggtggtggccatcgccagccacgatggcggcaagcaggctttggagacggtacagcggctgttgccggtgctgtgccaggcacatggcctgaccccggcccaagtggtggccatcgccagcaatggcggcggcaagcaggctctggagacggtacagcggctgttgccggtgctgtgccaggaccatggcctgaccccggaccaggtggtggccatcgccaacaataacggcggcaagcaggcgttggagacggtacagcggctgttgccggtgctgtgccaggaccatggcctgaccccggaccaggtcgtggccatcgccagcaatggcggcggcaagcaggctttggagacggtacagcggctgttgccggtgctgtgccaggaccatggcctgaccccggaccaggtcgtggccatcgccagcaatggcggcggcaagcaggcattggagacggtacagcggctgttgccggtgctgtgccaggaccatggcctgaccccggaccaggtcgtggccatcgccaacaataacggcggcaagcaggccctggagacggtacagcggctgttgccggtgctgtgccaggaccatggcctgaccccggaccaggtggtggccatcgccagcaatattggcggcaagcaggcactggagacggtacagcggctgttgccggtgctgtgccaggaccatggcctgaccccggaccaggtggtggccatcgccagccacgatggcggcaagcaggcactggaaacactgcagcggctgttgccggtgctgtgccaggaccatggcctgaccccggaccaggtggtggccatcgccagccacgatggcggcaagcaggccttggagacggtgcagcggctgttgccggtgctgtgccaggaccatggcctgaccccggaccaggtggtggccatcgccagccacgatggcggcaagcaggctctggagacggtacagcggctgttgccggtgctgtgccaggaccatggcctgaccccggaccaggtcgtggccatcgccagcaatattggcggcaagcaggcgttggagacggtacagcggctgttgccggtgctgtgccaggaccatggcctgaccccggaccaggtcgtggccatcgccagcaatattggcggcaagcaggctttggagacggtacagcggctgttgccggtgctgtgccaggaccatggcctgaccccggaccaggtcgtggccatcgccagcaatggcggcggcaagcaggcattggagacggtacagcggctgttgccggtgctgtgccaggaccatggcctgaccccggaccaggtggtggccatcgccaacaataacggcggcaagcaggccctggagacggtacagcggctgttgccggtgctgtgccaggaccatggcctgaccccggaccaggtggtggccatcgccagccacgatggcggcaagcaggcactggagacggtacagcggctgttgccggtgctgtgccaggaccatggcctgaccccgaaccaggtggtggccatcgccagcaatggcggcaagcaggcgctggagagcattgttgcccagttatctcgccctgatccggcgttggccgcgttgaccaacgaccacctcgtcgccttggcctgcctcggcggacgtcctgccctggatgcagtgaaaaagggattgccgcacgcgccggaattgatcagaagaatcaatcgccgcattcccgaacgcacgtcccatcgcgttcccgacctcgcgcacgtggttcgcgtgcttggttttttccagagccactcccacccagcgcaagcattcgatgacgccatgacgcagttcgagatgagcaggcacggcttggtacagctctttcgcagagtgggcgtcaccgaattcgaagcccgctacggaacgctccccccagcctcgcagcgttgggaccgtatcctccaggcatcagggatgaaaagggccaaaccgtcccctacttcagctcaaacaccggatcaggcgtctttgcatgcagattacaaggacgacgacgacaagaaggattacaaggacgacgacgacaagaagggtcgacccagcccaatgcacgagggagatcagacgcgggcaagcagccgtaaacggtcccgatcggatcgtgctgtcaccggcccctccacacagcaatctttcgaggtgcgcgttcccgaacagcaagatgcgctgcatttgcccctcagctggagggtaaaacgcccgcgtaccaggatcgggggcggcctcccggatcctggtacgcccatcgctgccgacctggcagcgtccagcaccgtgatgtgggaacaagatgcggcccccttcgcaggggcagcggatgatttcccggcattcaacgaagaggagctcgcatggttgatggagctattgcctcagtcaggctcagtcggagggacgatcaagcttctagactag

SEQ ID NO: 11 - DNA, 17 bp, Artificial sequence
  fusion of I-TevI from Bacteriophage T4 and Uv3b and EBE binding site from Xanthomonas oryzae
tactgttgacccaatgc

SEQ ID NO: 12 - DNA, 811 bp, Bacteriophage T4
atgaaaagcggaatttatcagattaaaaatactttaaacaataaagtatatgtaggaagtgctaaagattttgaaaagagatggaagaggcattttaaagatttagaaaaaggatgccattcttctataaaacttcagaggtcttttaacaaacatggtaatgtgtttgaatgttctattttggaagaaattccatatgagaaagatttgattattgaacgagaaaatttttggattaaagagcttaattctaaaattaatggatacaatattgctgatgcaacgtttggtgatacatgttctacgcatccattaaaagaagaaattattaagaaacgttctgaaactgttaaagctaagatgcttaaacttggacctgatggtcggaaagctctttacagtaaacccggaagtaaaaacgggcgttggaatccagaaacccataagttttgtaagtgcggtgttcgcatacaaacttctgcttatacttgtagtaaatgcagaggatccggaatcactagtagagaagccctacgcttgcccagtggagtcctgtgatcgccgcttctcccgctccgacgagctcacccgccacatccgcatccacacaggccagaagcccttccagtgccgcatctgcatgcgcaacttcagccgcagcgaccacctcaccacccacatccgcacccacacaggcgaaaagccctttgcctgcgacatctgtggaagaaagtttgccaggagcgatgaacgcaagaggcataccaagatccacggatccggaatcactagtctcgagtcatgtaattag

SEQ ID NO: 13 - protein, 92 aa, Bacteriophage T4
MKSGIYQIKNTLNNKVYVGSAKDFEKRWKRHFKDLEKGCHSSIKLQRSFNKHGNVFECSILEEIPYEKDLIIERENFWIKELNSKINGYNIA

SEQ ID NO: 14 - protein, 22 aa, Bacteriophage T4
DATFGDTCSTHPLKEEIIKKRS

SEQ ID NO: 15 - protein, 36 aa, Bacteriophage T4
ETVKAKMLKLGPDGRKALYSKPGSKNGRWNPETHKF

SEQ ID NO: 16 - protein, 18 aa, Bacteriophage T4
CKCGVRIQTSAYTCSKCR

SEQ ID NO: 17 - protein, 77 aa, Bacteriophage T4
NRSGENNSFFNHKHSDITKSKISEKMKGKKPSNIKKISCDGVIFDCAADAARHFKISSGLVTYRVKSDKWNWFYINA

SEQ ID NO: 18 - DNA, 313 bp, Artificial sequence
  fustion protein edited sequence
atgcatctccccctactgtacaccaccaaaagtgaattcatgagcttagcacctggttggagggggtttatatcgaaagctacatataaggaacgtgctgctactcatcctagtcctgttgctgccaagctatttaatatcatgcacgaaaagcaaacaaacttgtgtgcttcattggatgttcgtaccaccaaggaattactggagttagttgaagcattaggtcccaaaatttgtttactaaaaacacatgtggatatcttgactgatttttccatatggagggcacagttaagccgctaaaggcattatc

SEQ ID NO: 19 - DNA, 873 bp, Xanthomonas orryzae
atgcatctccccctactgtacaccaccaaaagtgaattcatgagcttagcacctggttggagggggtttatatcgaaagctacatataaggaacgtgctgctactcatcctagtcctgttgctgccaagctatttaatatcatgcacgaaaagcaaacaaacttgtgtgcttcattggatgttcgtaccaccaaggaattactggagttagttgaagcattaggtcccaaaatttgtttactaaaaacacatgtggatatcttgactgatttttccatggagggcacagttaagccgctaaaggcattatccgccaagtacaattttttactcttcgaagacagaaaatttgctgacattggtaatacagtcaaattgcagtactctgcgggtgtatacagaatagcagaatgggcagacattacgaatgcacacggtgtggtgggcccaggtattgttagcggtttgaagcaggcggcggaagaagtaacaaaggaacctagaggccttttgatgttagcagaattgtcatgcaagggctccctagctactggagaatatactaagggtactgttgacattgcgaagagcgacaaagattttgttatcggctttattgctcaaagagacatgggtggaagagatgaaggttacgattggttgattatgacacccggtgtgggtttagatgacaagggagacgcattgggtcaacagtatagaaccgtggatgatgtggtctctacaggatctgacattattattgttggaagaggactatttgcaaagggaagggatgctaaggtagagggtgaacgttacagaaaagcaggctgggaagcatatttgagaagatgcggccagcaaaactaa

SEQ ID NO: 20 - DNA, 469 bp, Artificial sequence
  DNA edited sequence
agtaacaaaaggaacctagaggccttttgatgttagcagaattgtcatgcaagggctccctagctactggagaatatactaagggtactgttgacattgcgaagagcgacaaagattttgttatcggctttattgctcaaagagacatgggtggaagagatgaaggttacgattggttgattatgacacccggtgtgggtttagatgacaagggagacgcattgggtcaacagtagtaacaaaaaggaacctagaggccttttgatgttagcagaattgtcatgcaagggctccctagctactggagaatatactaagggtactgttgacattgcgaagagcgacaaagattttgttatcggctttattgctcaaagagacatgggtggaagagatgaaggttacgattggttgattatgacacccggtgtgggtttagatgacaagggagacgcattgggtcaacagt

SEQ ID NO: 21 - DNA, 873 bp, Xanthomonas oryzae; identical to SEQ ID NO: 19

(19) United States
(12) Patent Application Publication
(10) Pub. No.: US 2015/0017728 A1
(43) Pub. Date: Jan. 15, 2015

(54) MONOMER ARCHITECTURE OF TAL NUCLEASE OR ZINC FINGER NUCLEASE FOR DNA MODIFICATION

(71) Applicant: IOWA STATE UNIVERSITY RESEARCH FOUNDATION, INC, Ames, IA (US)

(72) Inventors: Bing Yang, Ames, IA (US); Ting Li, Ames, IA (US); Sheng Huang, Ames, IA (US)

(21) Appl. No.: 14/344,526

(22) PCT Filed: Sep. 19, 2012
(86) PCT No.: PCT/US12/55980

§ 371 Date: Mar. 12, 2014

Related U.S. Application Data
(60) Provisional application No. 61/538,260, filed on Sep. 23, 2011.

(51) Int. Cl. C12N 15/90; C12N 9/22

(52) U.S. Cl. CPC C12N 15/905; C12N 9/22; C12N 2800/80; C07K 2319/81
USPC 435/462; 435/325; 435/468; 435/471; 435/419; 435/254.11; 435/320.1; 435/199; 536/23.2

(57) ABSTRACT

The present invention provides compositions and methods for targeted cleavage of cellular chromatin in a region of interest and/or homologous recombination at a predetermined site in cells. Compositions include fusion polypeptides comprising a TAL effector binding or a zinc finger domain and an I-TevI homing endonuclease cleavage domain as well as nucleic acid sequence encoding the same. The use of the I-TevI domain allows for monomer endonuclease sequences to achieve cleavage of cellular chromatin and represents an advantage over prior endonucleases which require self-dimerization, and two nucleases with appropriate spacers.

### CROSS-REFERENCE TO RELATED APPLICATIONS

This application claims priority under 35 U.S.C. §119 to provisional application Ser. No. 61/538,260 filed Sep. 23, 2011, herein incorporated by reference in its entirety.

### STATEMENT AS TO FEDERALLY SPONSORED RESEARCH

This invention was made with government support under Grant No. DBI-0820831 awarded by the US National Science Foundation. The government has certain rights in the invention.

### TECHNICAL FIELD

This invention relates to methods for DNA modification, such as homologous recombination and gene targeting, and particularly to methods that include the use of fusion proteins with transcription activator-like (TAL) effector sequences or zinc finger motifs with DNA cleavage domains of nucleases.

### BACKGROUND OF THE INVENTION

DNA double-strand breaking (DSB) enhances homologous recombination in living cells and has been exploited for targeted genome editing through use of engineered endonucleases, notably zinc finger nucleases (ZFN), a type of hybrid enzyme consisting of DNA binding domains of zinc finger proteins and the FokI nuclease domain (FN). Similarly, nucleases can also be made by using other proteins/domains if they are capable of specific DNA recognition.

The most significant application of endonucleases that are modified or custom-engineered to recognize longer DNA sequences is target genome editing in the post-genome era. The key component of the engineered nucleases is the DNA recognition domain that is capable of directing the nuclease to the target site of genome for a genomic DNA double strand break. The cellular DSB repair due to nonhomologous end-joining (NHEJ) results in mutagenic deletions/insertions of a target gene. Alternately, the DSB can stimulate homologous recombination between the endogenous target locus and an exogenously introduced homologous DNA fragment with desired genetic information, a process called gene targeting. The most promising method involving gene or genome editing is the custom-designed ZFN technology. The ZFN technology primarily involves the use of hybrid proteins derived from the DNA binding domains of zinc finger (ZF) proteins and the nonspecific cleavage domain of the endonuclease FokI. The ZFs can be assembled as modules that are custom-designed to recognize selected DNA sequences following binding at the preselected site, a DSB is produced by the action of cleavage domain of FokI.

The FokI endonuclease was first isolated from the bacterium Flavobacterium okeanokoites. This type IIS nuclease consists of two separate domains, the N-terminal DNA binding domain and C-terminal DNA cleavage domain. The DNA binding domain functions for recognition of a non-palindromic sequence 5′-GGATG-3′/5′-CATCC-3′ while the catalytic domain cleaves double-stranded DNA non-specifically at a fixed distance of 9 and 13 nucleotides downstream of the recognition site. FokI exists as an inactive monomer in solution and becomes an active dimmer following the binding to its target DNA and in the presence of some divalent metals. As a functional complex, two molecules of FokI each binding to a double stranded DNA molecule dimerize through the DNA catalytic domain for the effective cleavage of DNA double strands.

ZFN technology has been successfully applied for genetic modification to a variety of organisms, including yeast, plants, fungi and mammals, and even human cell lines. Despite the promise of ZFN technology, however, widespread adoption of this technology is hampered by a bottleneck in custom-engineering zinc fingers capable of high specificity and affinity for the target sites, a process that is labor intensive and associated with high rate of failures. The essence of these endonucleases lies on the DNA binding specificity, which theoretically can be supplanted by any DNA binding proteins/domains when fused with an endonuclease domain, such as a group of TAL effector proteins from bacterial plant pathogens of Xanthomonas.

TAL effectors belong to a large group of bacterial proteins that exist in various strains of Xanthomonas spp. and are translocated into host cells by a type III secretion system, so called type III effectors. Once in host cells, some TAL effectors have been found to transcriptionally activate their corresponding host target genes either for strain virulence (ability to cause disease) or avirulence (capacity to trigger host resistance responses) dependent on the host genetic context. Each effector contains the functional nuclear localization motifs and a potent transcription activation domain that are characteristic of eukaryotic transcription activator. And each effector also contains a central repetitive region consisting of varying numbers of repeat units of 34 amino acids, and the repeat region as DNA binding domain determines the biological specificity of each effector. The repeat is nearly identical except for the variable amino acids at positions 12 and 13, so called repeat variable di-residues (RVD), of each repeat. Recent studies have revealed the recognition of DNA sequences within the promoters of host target genes by the repeat regions of TAL effectors, and the recognition could be simplified in a code that one nucleotide of a target site is corresponding in a sequential order to the RVD of one repeat, with the tandem array of repeats corresponding to a specific, consecutive stretch of DNA. The majority of naturally occurring TAL proteins contain repeat units in a range of 13 to 29 repeats that presumably recognize DNA elements consisting of same number of nucleotides. Furthermore, the so called TAL recognition code could be used to guide the custom-design of novel TAL proteins or repeats with an array of repeat units that can function as DNA binding motifs for a specific and constitutive sequential DNA sequence although such feasibility needs to be determined.

### SUMMARY OF THE INVENTION

The present invention provides compositions and methods for targeted cleavage of cellular chromatin in a region of interest and/or homologous recombination at a predetermined region of interest in cells. Cells include cultured cells, cells in an organism and cells that have been removed from an organism for treatment in cases where the cells and/or their descendants will be returned to the organism after treatment. A region of interest in cellular chromatin can be, for example, a genomic sequence or portion thereof. Compositions include fusion polypeptides comprising a TAL effector binding domain or a zinc finger binding domain and a cleavage domain. The novel cleavage domain disclosed herein is the use of the I-TevI homing nuclease. The use of Type IIS restriction endonucleases, such as FokI and uses of the fusion proteins is disclosed in U.S. patent application Ser. No. 13/025,405 filed Feb. 11, 2011, and United State Published Application 2010/0214228 particularly paragraphs 78-192 the disclosure of each is hereby incorporated in its entirety by reference.

The nature of FokI nuclease domain requires self-dimerization for cutting DNA such that two engineered nucleases (a dimer) must be present at the specific site with one nuclease binding to one strand and the other to an adjacent site of the opposite strand. The two binding sites must be appropriately separated so the two FokI cleavage domains (one from each engineered nuclease) can dimerize and cause double strand break (DSB). There are a number of limitations to its applicability for this kind of architecture. For example, two nucleases have to be made for each target site of interest; the overall cleaving efficiency will depend on the coordination of these two nucleases; requirement of two sites and appropriate length of spacer collectively limit the choice of potential target sites for engineering suitable nucleases. Therefore, it is desirable to develop an architecture that enables single engineered nucleases to be functional as efficiently as dimeric nucleases, here in case of TALENs and ZFNs.

Applicants have identified that the DNA cleavage domain of homing nuclease I-TevI is amenable to fusion with TAL effector proteins and zinc finger proteins with a monomer architecture. The fusion proteins exhibited the DNA binding specificity of ZF and TALEs and DNA cleaving activity to double strands near the binding sites as monomer. Both monomeric ZFNs and TALENs when expressed in yeast cells were able to induce DSBs in the plasmid carrying the respective target sequences and stimulate the homologous recombination of two duplicated regions in the yeast single strand annealing (SSA) assay. When expressed in yeast cells, the monomeric nucleases induced gene disruption at the expected sites where single stretches of the target DNA sequences were present. Taken together, the I-TevI DNA cleavage domain and the TALEs and ZFPs can be linked to form active monomeric nucleases capable of modifying genes at specific sites.

Cellular chromatin to be modified according to the invention can be present in any type of cell including, but not limited to, prokaryotic and eukaryotic cells, fungal cells, plant cells, animal cells, mammalian cells, primate cells and human cells. Cellular chromatin can be present, e.g., in chromosomes or in intracellular genomes of infecting bacteria or viruses.

Thus the invention comprises a method for modifying the genetic material of a cell. The method includes providing a primary cell containing a chromosomal target DNA sequence in which it is desired to have homologous recombination occur, providing a TAL effector endonuclease comprising an endonuclease domain that can cleave double stranded DNA, and a TAL effector domain comprising a plurality of TAL effector repeat sequences that, in combination, bind to a specific nucleotide sequence within the target DNA in the cell; and contacting the target DNA sequence with the TAL effector endonuclease in the cell such that the TAL effector endonuclease cleaves both strands of a nucleotide sequence within or adjacent to the target DNA sequence in the cell. The method can further include providing a nucleic acid comprising a sequence homologous to at least a portion of the target DNA, such that homologous recombination occurs between the target DNA sequence and the nucleic acid. The target DNA sequence can be endogenous to the cell. The cell can be a plant cell or a mammalian cell. The contacting can include transfecting the cell with a vector comprising a TAL effector endonuclease coding sequence, and expressing the TAL effector endonuclease protein in the cell, mechanically injecting a TAL effector endonuclease protein into the cell, delivering a TAL effector endonuclease protein into the cell by means of the bacterial type III secretion system, or introducing a TAL effector endonuclease protein into the cell by electroporation. The endonuclease domain is from ITev-I. The TAL effector domain that binds to a specific nucleotide sequence within the target DNA can include 15 or more DNA binding repeats. The cell can be from an organism selected from the group consisting of a plant, an animal, a mammal, a human, a teleost fish, a fungus, a bacteria or a protozoa.

In another embodiment the invention includes a method for designing a fusion protein with a sequence specific TAL effector endonuclease or zinc finger endonuclease either of which is capable of cleaving DNA at a specific location using the novel I-TevI nuclease fused to an appropriate target binding domain. The method includes identifying a first unique endogenous chromosomal nucleotide sequence adjacent to a second nucleotide sequence at which it is desired to introduce a double-stranded cut; and designing a sequence specific TAL effector endonuclease comprising (a) a plurality of DNA binding repeat domains that, in combination, bind to the first unique endogenous chromosomal nucleotide sequence, or an appropriate zinc finger domain that binds to the first unique endogenous chromosomal nucleotide sequence and (b) an I-TevI endonuclease that generates a double-stranded cut at the second nucleotide sequence.

According to the invention, the fusion protein can be expressed in a cell, e.g., by delivering the fusion protein to the cell or by delivering a polynucleotide encoding the fusion protein to a cell, wherein the polynucleotide, if DNA, is transcribed, and an RNA molecule delivered to the cell or a transcript of a DNA molecule delivered to the cell is translated, to generate the fusion protein. Methods for polynucleotide and polypeptide delivery to cells are known in the art and are presented elsewhere in this disclosure.

Targeted mutations resulting from the aforementioned method include, but are not limited to, point mutations (i.e., conversion of a single base pair to a different base pair), substitutions (i.e., conversion of a plurality of base pairs to a different sequence of identical length), insertions or one or more base pairs, deletions of one or more base pairs and any combination of the aforementioned sequence alterations.

Methods for targeted recombination (for, e.g., alteration or replacement of a sequence in a chromosome or a region of interest in cellular chromatin) are also provided. For example, a mutant genomic sequence can be replaced by a wild-type sequence, e.g., for treatment of genetic disease or inherited disorders. In addition, a wild-type genomic sequence can be replaced by a mutant sequence, e.g., to prevent function of an oncogene product or a product of a gene involved in an inappropriate inflammatory response. Furthermore, one allele of a gene can be replaced by a different allele.

The invention also includes a TAL effector endonuclease comprising a I-Tev endonuclease domain and a TAL effector or ZFN DNA binding domain specific for a particular DNA sequence. The TAL effector endonuclease can further include a purification tag.

This invention provides a I-TevI polypeptide fragment derived from I-TevI and capable of performing monomeric cleavage when combined with a ZFN or TAL effector binding domain and having endonuclease activity comprising (a) a polypeptide comprising at least 90% homology, more preferably at least 95% homology, or more preferably at least 96%, 97%, 98%, or 99% sequence identity to a polypeptide of SEQ ID NO:2 (b) a polypeptide encoded by a nucleic acid of the present invention; and (c) a conservatively modified variant thereof.

The invention also includes fusion proteins made by combining I-TevI sequences with ZFN or TAL effector domains.

In another aspect of the invention, the invention comprises a nucleic acids encoding the I-TevI domain and fusion proteins above (a) encoding an I-TevI domain of SEQ ID NO:2; or (b) having a nucleic acid sequence of SEQ ID NO:3, or (c) having a nucleic acid sequence which is at least 90% homology, more preferably at least 95% homology, or more preferably at least 96%, 97%, 98%, or 99% homology to one of SEQ ID NO:3, or (d) which hybridizes to a nucleic acid sequence which encodes an I-TevI domain under at least conditions of high stringency.

The invention includes nucleic acid sequences and resulting fusion proteins of I-TevI sequences and ZFN or TAL effector sequences. In some embodiments the TAL effector sequences I AvrXa7, or is SEQ ID NO:6 and includes the EBE of SEQ ID NO:7. In some embodiments the includes nucleic acid sequences and resulting fusion proteins of I-Tev domain and the Zinc finger protein sequence of Zif268 or is SEQ ID NO 12.

### DESCRIPTION OF THE FIGURES

FIG. 1 shows the structure of monomer TAL nuclease. Full-length TAL effector (TALE) (e.g., AvrXa7) is fused at the C-terminus of endonuclease Tev-I (Tv) (N-terminal 168 amino acid DNA cleavage domain of Tev-I). The Tv is linked through restriction site BamHI with TAL effector proteins which contain the varying number of repeats.

FIG. 2 shows TevI-AvrXa7 nuclease activities on plasmid DNA. Agarose gel image of plasmid DNA pTOPO/1N3 (containing effector binding element, EBE, of AvrXa7) treated with restriction enzyme MluI and subsequently the purified recombinant Tv-AvrXa7. MluI completely cuts pTOPO/11N3 into two fragments (2.05 kb and 1.0 kb as indicated by the arrows). The 2.05 kb AvrXa7-EBE-containing fragment is cleaved by Tv-AvrXa7 into 1.42 and 0.63 kb as expected (1.42 and 0.63 kb). Lane 1, 1 kb marker, lanes 2, 3, 4 represent the same amount (250 ng) of DNA treated with different concentrations (0.25 ug, 0.5 ug, and 0.75 ug) of Tv-AvrXa7.

FIG. 3 shows Tv-AvrXa7 nuclease activity in linearizing plasmid DNA. A gel image of plasmid pTOPO/11N3 treated with Tv-AvrXa7. Lower band is the super coiled plasmid. The upper band is the linearized plasmid. Lane 1 is 1 kb marker. Lane 2-7 is the plasmid pTOPO/11N3 treated with 0, 0.1, 0.2, 0.3, 0.4, 0.5 ug of TvI-AvrXa7 individually.

FIG. 4 shows homologous recombination of plasmid-borne reporter gene in yeast mediated by monomer TALENs. (a) Constructs of the reporter gene LacZ. Two LacZ fragments (LacZN and LacZC) sharing a duplicated 125 bp portion (hatched boxes) of LacZ coding region were separated by a sequence of the respective TAL effector or zinc finger protein binding sites (AvrXa7 EBE, U3b-R EBE, or Zif268 EBE). (b) Yeast colonies stained with the substrate (X-gal, 5-bromo-4-chloro-3-indolyl-β-d-galactopyranoside) of the LacZ gene product (β-galactosidase). The color density of colonies in blue reflects the activities of monomer TAL nucleases in cleaving and stimulating the homologous recombination of the two duplicated LacZ regions of the reporter constructs in yeast cells. Yeast colonies with effector construct (control, empty vector lacking any nuclease gene; Tv-avrXa7, Tv-U3b-R, and Tv-Zif268) and their respective target sequences were transferred through colony-lift onto filter membrane and stained with X-gal for 2 hrs and photographed.

FIG. 5 is the amino acid sequence of I-TevI homing endonuclease, GenBank accession number, AAD42521. Amino acids 1-92 (underlined) is the catalytic GIY-YIG domain; 93-114 as (shaded in grey) is the deletion intolerant domain; 115-149 as (italic) is the deletion tolerant domain; 150-168 as (boxed) is the zinc finger domain; 169-254 as (in bold black) is the DNA binding domain. N-terminus of 168 amino acids (Tv) is used to fuse with either TAL effectors or zinc finger proteins for monomer TAL nucleases or monomer zinc finger nucleases.

FIG. 6 shows the results of sequencing of 5-FOA resistant yeast colonies. 6(a) Tv-AvrXa7-Four colonies were genotyped and were found to contain the same mutation illustrated. The underlined 26 base pairs were the binding site of AvrXa7, the red lower “at” is the inserted sequence which caused the frame shift of URA3 open reading frame (thus, mutagenic and conferring resistance to 5-FOA). 6(b) is the functional URA3 coding sequence.

FIG. 7 shows the results of sequencing. Eight colonies were genotyped and seven were found to contain an “a” insertion and one to contain an “aa” insertion. 7(a) Tv-U3b-R The mutations are red and lower cases. 7(b) is the functional URA3 coding sequence.

### DETAILED DESCRIPTION OF THE INVENTION

### General

Practice of the methods, as well as preparation and use of the compositions disclosed herein employ, unless otherwise indicated, conventional techniques in molecular biology, biochemistry, chromatin structure and analysis, computational chemistry, cell culture, recombinant DNA and related fields as are within the skill of the art. These techniques are fully explained in the literature. See, for example, Sambrook et al. MOLECULAR CLONING: A LABORATORY MANUAL, Second edition, Cold Spring Harbor Laboratory Press, 1989 and Third edition, 2001; Ausubel et al., CURRENT PROTOCOLS IN MOLECULAR BIOLOGY, John Wiley & Sons, New York, 1987 and periodic updates; the series METHODS IN ENZYMOLOGY, Academic Press, San Diego; Wolffe, CHROMATIN STRUCTURE AND FUNCTION, Third edition, Academic Press, San Diego, 1998; METHODS IN ENZYMOLOGY, Vol. 304, “Chromatin” (P. M. Wassarman and A. P. Wolffe, eds.), Academic Press, San Diego, 1999; and METHODS IN MOLECULAR BIOLOGY, Vol. 119, “Chromatin Protocols” (P. B. Becker, ed.) Humana Press, Totowa, 1999.

### DEFINITIONS

“Binding” refers to a sequence-specific, non-covalent interaction between macromolecules (e.g., between a protein and a nucleic acid). Not all components of a binding interaction need be sequence-specific (e.g., contacts with phosphate residues in a DNA backbone), as long as the interaction as a whole is sequence-specific. Such interactions are generally characterized by a dissociation constant (K_{d}) of 10⁻⁶ M⁻¹ or lower. “Affinity” refers to the strength of binding: increased binding affinity being correlated with a lower K_{d}.

A “binding protein” is a protein that is able to bind non-covalently to another molecule. A binding protein can bind to, for example, a DNA molecule (a DNA-binding protein), an RNA molecule (an RNA-binding protein) and/or a protein molecule (a protein-binding protein). In the case of a protein-binding protein, it can bind to itself (to form homodimers, homotrimers, etc.) and/or it can bind to one or more molecules of a different protein or proteins. A binding protein can have more than one type of binding activity. For example, zinc finger proteins have DNA-binding, RNA-binding and protein-binding activity.

A “TAL effector DNA binding protein” (or binding domain) or a “TAL effector DNA recognition sequence” is a protein encompassing a series of repeat variable-diresidues (RVDs) within a larger protein, that binds DNA in a sequence-specific manner. The RVD regions of TAL effectors are polymorphisms within TALs typically at positions 12 and 13 in repeating units of typically 34 amino acids that bind for specific nucleotides and together with a plurality of repeating unit intervals make up the specific TAL effector DNA binding domain.

TAL effector DNA binding protein domains (their RVDs) can be “engineered” to bind to a predetermined nucleotide sequence. Non-limiting examples of methods for engineering the same are design and selection. A designed TAL effector DNA binding protein is a protein not occurring in nature whose design/composition results principally from rational criteria. Rational criteria for design include application of substitution rules and computerized algorithms for processing information in a database storing information of existing RVD designs and binding data.

The term “sequence” refers to a nucleotide sequence of any length, which can be DNA or RNA; can be linear, circular or branched and can be either single-stranded or double stranded. The term “donor sequence” refers to a nucleotide sequence that is inserted into a genome. A donor sequence can be of any length, for example between 2 and 10,000 nucleotides in length (or any integer value there between or thereabove), preferably between about 100 and 1,000 nucleotides in length (or any integer there between), more preferably between about 200 and 500 nucleotides in length.

“Recombination” refers to a process of exchange of genetic information between two polynucleotides. For the purposes of this disclosure, “homologous recombination (HR)” refers to the specialized form of such exchange that takes place, for example, during repair of double-strand breaks in cells. This process requires nucleotide sequence homology, uses a “donor” molecule to template repair of a “target” molecule (i.e., the one that experienced the double-strand break), and is variously known as “non-crossover gene conversion” or “short tract gene conversion,” because it leads to the transfer of genetic information from the donor to the target. Without wishing to be bound by any particular theory, such transfer can involve mismatch correction of heteroduplex DNA that forms between the broken target and the donor, and/or “synthesis-dependent strand annealing,” in which the donor is used to resynthesize genetic information that will become part of the target, and/or related processes. Such specialized HR often results in an alteration of the sequence of the target molecule such that part or all of the sequence of the donor polynucleotide is incorporated into the target polynucleotide.

“Cleavage” refers to the breakage of the covalent backbone of a DNA molecule. Cleavage can be initiated by a variety of methods including, but not limited to, enzymatic or chemical hydrolysis of a phosphodiester bond. Both single-stranded cleavage and double-stranded cleavage are possible, and double-stranded cleavage can occur as a result of two distinct single-stranded cleavage events. DNA cleavage can result in the production of either blunt ends or staggered ends. In certain embodiments, fusion polypeptides are used for targeted double-stranded DNA cleavage.

A “cleavage domain” comprises one or more polypeptide sequences which possesses catalytic activity for DNA cleavage. A cleavage domain can be contained in a single polypeptide chain or cleavage activity can result from the association of two (or more) polypeptides.

“Chromatin” is the nucleoprotein structure comprising the cellular genome. Cellular chromatin comprises nucleic acid, primarily DNA, and protein, including histones and non-histone chromosomal proteins. The majority of eukaryotic cellular chromatin exists in the form of nucleosomes, wherein a nucleosome core comprises approximately 150 base pairs of DNA associated with an octamer comprising two each of histones H2A, H2B, H3 and H4; and linker DNA (of variable length depending on the organism) extends between nucleosome cores. A molecule of histone H1 is generally associated with the linker DNA. For the purposes of the present disclosure, the term “chromatin” is meant to encompass all types of cellular nucleoprotein, both prokaryotic and eukaryotic. Cellular chromatin includes both chromosomal and episomal chromatin.

A “chromosome,” is a chromatin complex comprising all or a portion of the genome of a cell. The genome of a cell is often characterized by its karyotype, which is the collection of all the chromosomes that comprise the genome of the cell. The genome of a cell can comprise one or more chromosomes.

An “accessible region” is a site in cellular chromatin in which a target site present in the nucleic acid can be bound by an exogenous molecule which recognizes the target site. Without wishing to be bound by any particular theory, it is believed that an accessible region is one that is not packaged into a nucleosomal structure. The distinct structure of an accessible region can often be detected by its sensitivity to chemical and enzymatic probes, for example, nucleases.

A “target site” or “target sequence” is a nucleic acid sequence that defines a portion of a nucleic acid to which a binding molecule will bind, provided sufficient conditions for binding exist. For example, the sequence 5′-GAATTC-3′ is a target site for the Eco RI restriction endonuclease.

An “exogenous” molecule is a molecule that is not normally present in a cell, but can be introduced into a cell by one or more genetic, biochemical or other methods. “Normal presence in the cell” is determined with respect to the particular developmental stage and environmental conditions of the cell. Thus, for example, a molecule that is present only during embryonic development of muscle is an exogenous molecule with respect to an adult muscle cell. Similarly, a molecule induced by heat shock is an exogenous molecule with respect to a non-heat-shocked cell. An exogenous molecule can comprise, for example, a functioning version of a malfunctioning endogenous molecule or a malfunctioning version of a normally-functioning endogenous molecule.

An exogenous molecule can be, among other things, a small molecule, such as is generated by a combinatorial chemistry process, or a macromolecule such as a protein, nucleic acid, carbohydrate, lipid, glycoprotein, lipoprotein, polysaccharide, any modified derivative of the above molecules, or any complex comprising one or more of the above molecules. Nucleic acids include DNA and RNA, can be single- or double-stranded; can be linear, branched or circular; and can be of any length. Nucleic acids include those capable of forming duplexes, as well as triplex-forming nucleic acids. See, for example, U.S. Pat. Nos. 5,176,996 and 5,422,251. Proteins include, but are not limited to, DNA-binding proteins, transcription factors, chromatin remodeling factors, methylated DNA binding proteins, polymerases, methylases, demethylases, acetylases, deacetylases, kinases, phosphatases, integrases, recombinases, ligases, topoisomerases, gyrases and helicases.

An exogenous molecule can be the same type of molecule as an endogenous molecule, e.g., an exogenous protein or nucleic acid. For example, an exogenous nucleic acid can comprise an infecting viral genome, a plasmid or episome introduced into a cell, or a chromosome that is not normally present in the cell. Methods for the introduction of exogenous molecules into cells are known to those of skill in the art and include, but are not limited to, lipid-mediated transfer (i.e., liposomes, including neutral and cationic lipids), electroporation, direct injection, cell fusion, particle bombardment, calcium phosphate co-precipitation, DEAE-dextran-mediated transfer and viral vector-mediated transfer.

By contrast, an “endogenous” molecule is one that is normally present in a particular cell at a particular developmental stage under particular environmental conditions. For example, an endogenous nucleic acid can comprise a chromosome, the genome of a mitochondrion, chloroplast or other organelle, or a naturally-occurring episomal nucleic acid. Additional endogenous molecules can include proteins, for example, transcription factors and enzymes.

A “fusion” molecule is a molecule in which two or more subunit molecules are linked, preferably covalently. The subunit molecules can be the same chemical type of molecule, or can be different chemical types of molecules. Examples of the first type of fusion molecule include, but are not limited to, fusion proteins (for example, a fusion between a TAL effector sequence DNA-binding domain, or zinc finger domain and a cleavage domain) and fusion nucleic acids (for example, a nucleic acid encoding the fusion protein described supra). Examples of the second type of fusion molecule include, but are not limited to, a fusion between a triplex-forming nucleic acid and a polypeptide, and a fusion between a minor groove binder and a nucleic acid.

Expression of a fusion protein in a cell can result from delivery of the fusion protein to the cell or by delivery of a polynucleotide encoding the fusion protein to a cell, wherein the polynucleotide is transcribed, and the transcript is translated, to generate the fusion protein. Trans-splicing, polypeptide cleavage and polypeptide ligation can also be involved in expression of a protein in a cell. Methods for polynucleotide and polypeptide delivery to cells are presented elsewhere in this disclosure.

A “gene,” for the purposes of the present disclosure, includes a DNA region encoding a gene product (see infra), as well as all DNA regions which regulate the production of the gene product, whether or not such regulatory sequences are adjacent to coding and/or transcribed sequences. Accordingly, a gene includes, but is not necessarily limited to, promoter sequences, terminators, translational regulatory sequences such as ribosome binding sites and internal ribosome entry sites, enhancers, silencers, insulators, boundary elements, replication origins, matrix attachment sites and locus control regions.

“Gene expression” refers to the conversion of the information, contained in a gene, into a gene product. A gene product can be the direct transcriptional product of a gene (e.g., mRNA, tRNA, rRNA, antisense RNA, ribozyme, structural RNA or any other type of RNA) or a protein produced by translation of a mRNA. Gene products also include RNAs which are modified, by processes such as capping, polyadenylation, methylation, and editing, and proteins modified by, for example, methylation, acetylation, phosphorylation, ubiquitination, ADP-ribosylation, myristilation, and glycosylation.

“Modulation” of gene expression refers to a change in the activity of a gene. Modulation of expression can include, but is not limited to, gene activation and gene repression.

“Eucaryotic” cells include, but are not limited to, fungal cells (such as yeast), plant cells, animal cells, mammalian cells and human cells.

A “region of interest” is any region of cellular chromatin, such as, for example, a gene or a non-coding sequence within or adjacent to a gene, in which it is desirable to bind an exogenous molecule. Binding can be for the purposes of targeted DNA cleavage and/or targeted recombination. A region of interest can be present in a chromosome, an episome, an organellar genome (e.g., mitochondrial, chloroplast), or an infecting viral genome, for example. A region of interest can be within the coding region of a gene, within transcribed non-coding regions such as, for example, leader sequences, trailer sequences or introns, or within non-transcribed regions, either upstream or downstream of the coding region. A region of interest can be as small as a single nucleotide pair or up to 2,000 nucleotide pairs in length, or any integral value of nucleotide pairs.

The terms “operative linkage” and “operatively linked” (or “operably linked”) are used interchangeably with reference to a juxtaposition of two or more components (such as sequence elements), in which the components are arranged such that both components function normally and allow the possibility that at least one of the components can mediate a function that is exerted upon at least one of the other components. By way of illustration, a transcriptional regulatory sequence, such as a promoter, is operatively linked to a coding sequence if the transcriptional regulatory sequence controls the level of transcription of the coding sequence in response to the presence or absence of one or more transcriptional regulatory factors. A transcriptional regulatory sequence is generally operatively linked in cis with a coding sequence, but need not be directly adjacent to it. For example, an enhancer is a transcriptional regulatory sequence that is operatively linked to a coding sequence, even though they are not contiguous.

With respect to fusion polypeptides, the term “operatively linked” can refer to the fact that each of the components performs the same function in linkage to the other component as it would if it were not so linked. For example, with respect to a fusion polypeptide in which a TAL effector DNA-binding domain is fused to a cleavage domain, the TAL effector DNA-binding domain and the cleavage domain are in operative linkage if, in the fusion polypeptide, the TAL effector DNA-binding domain portion is able to bind its target site and/or its binding site, while the cleavage domain is able to cleave DNA in the vicinity of the target site.

A “functional fragment” of a protein, polypeptide or nucleic acid is a protein, polypeptide or nucleic acid whose sequence is not identical to the full-length protein, polypeptide or nucleic acid, yet retains the same function as the full-length protein, polypeptide or nucleic acid. A functional fragment can possess more, fewer, or the same number of residues as the corresponding native molecule, and/or can contain one or more amino acid or nucleotide substitutions. Methods for determining the function of a nucleic acid (e.g., coding function, ability to hybridize to another nucleic acid) are well-known in the art. Similarly, methods for determining protein function are well-known. For example, the DNA-binding function of a polypeptide can be determined, for example, by filter-binding, electrophoretic mobility-shift, or immunoprecipitation assays. DNA cleavage can be assayed by gel electrophoresis. See Ausubel et al., supra. The ability of a protein to interact with another protein can be determined, for example, by co-immunoprecipitation, two-hybrid assays or complementation, both genetic and biochemical. See, for example, Fields et al. (1989) Nature 340:245-246; U.S. Pat. No. 5,585,245 and PCT WO 98/44350.

### DEFINITIONS

The term “conservatively modified variants” applies to both amino acid and nucleic acid sequences. With respect to particular nucleic acid sequences, conservatively modified variants refers to those nucleic acids which encode identical or conservatively modified variants of the amino acid sequences. Because of the degeneracy of the genetic code, a large number of functionally identical nucleic acids encode any given protein. For instance, the codons GCA, GCC, GCG and GCU all encode the amino acid alanine. Thus, at every position where an alanine is specified by a codon, the codon can be altered to any of the corresponding codons described without altering the encoded polypeptide. Such nucleic acid variations are “silent variations” and represent one species of conservatively modified variation. Every nucleic acid sequence herein that encodes a polypeptide also, by reference to the genetic code, describes every possible silent variation of the nucleic acid.

One of ordinary skill will recognize that each codon in a nucleic acid (except AUG, which is ordinarily the only codon for methionine; and UGG, which is ordinarily the only codon for tryptophan) can be modified to yield a functionally identical molecule. Accordingly, each silent variation of a nucleic acid which encodes a polypeptide of the present invention is implicit in each described polypeptide sequence and is within the scope of the present invention.

As to amino acid sequences, one of skill will recognize that individual substitutions, deletions or additions to a nucleic acid, peptide, polypeptide, or protein sequence which alters, adds or deletes a single amino acid or a small percentage of amino acids in the encoded sequence is a “conservatively modified variant” where the alteration results in the substitution of an amino acid with a chemically similar amino acid. Thus, any number of amino acid residues selected from the group of integers consisting of from 1 to 15 can be so altered. Thus, for example, 1, 2, 3, 4, 5, 7, or 10 alterations can be made.

Conservatively modified variants typically provide similar biological activity as the unmodified polypeptide sequence from which they are derived. For example, substrate specificity, enzyme activity, or ligand/receptor binding is generally at least 30%, 40%, 50%, 60%, 70%, 80%, or 90% of the native protein for its native substrate. Conservative substitution tables providing functionally similar amino acids are well known in the art.

The following six groups each contain amino acids that are conservative substitutions for one another: 1) Alanine (A), Serine (S), Threonine (T); 2) Aspartic acid (D), Glutamic acid (E); 3) Asparagine (N), Glutamine (Q); 4) Arginine (R), Lysine (K); 5) Isoleucine (I), Leucine (L), Methionine (M), Valine (V); and 6) Phenylalanine (F), Tyrosine (Y), Tryptophan (W). See also, Creighton (1984) Proteins W.H. Freeman and Company.

By “encoding” or “encoded”, with respect to a specified nucleic acid, is meant comprising the information for translation into the specified protein. A nucleic acid encoding a protein may comprise intervening sequences (e.g., introns) within translated regions of the nucleic acid, or may lack such intervening non-translated sequences (e.g., as in cDNA). The information by which a protein is encoded is specified by the use of codons. Typically, the amino acid sequence is encoded by the nucleic acid using the “universal” genetic code. However, variants of the universal code, such as are present in some plant/algae, animal, and fungal mitochondria, the bacterium Mycoplasma capricolum, or the ciliate Macronucleus, may be used when the nucleic acid is expressed therein. When the nucleic acid is prepared or altered synthetically, advantage can be taken of known codon preferences of the intended host where the nucleic acid is to be expressed.

As used herein “full-length sequence” in reference to a specified polynucleotide or its encoded protein means having the entire amino acid sequence of, a native (nonsynthetic), endogenous, biologically active form of the specified protein. Methods to determine whether a sequence is full-length are well known in the art including such exemplary techniques as northern or western blots, primer extension, S 1 protection, and ribonuclease protection. See, e.g., Plant Molecular Biology: A Laboratory Manual, Clark, Ed., Springer-Verlag, Berlin (1997). Comparison to known full-length homologous (orthologous and/or paralogous) sequences can also be used to identify full-length sequences of the present invention. Additionally, consensus sequences typically present at the 5′ and 3′untranslated regions of mRNA aid in the identification of a polynucleotide as full-length. For example, the consensus sequence ANNNNAUGG, where the underlined codon represents the N-terminal methionine, aids in determining whether the polynucleotide has a complete 5′end. Consensus sequences at the 3′end, such as polyadenylation sequences, aid in determining whether the polynucleotide has a complete 3′end.

As used herein, “heterologous” in reference to a nucleic acid is a nucleic acid that originates from a foreign species, or, if from the same species, is substantially modified from its native form in composition and/or genomic locus by deliberate human intervention. For example, a promoter operably linked to a heterologous structural gene is from a species different from that from which the structural gene was derived, or, if from the same species, one or both are substantially modified from their original form. A heterologous protein may originate from a foreign species or, if from the same species, is substantially modified from its original form by deliberate human intervention.

By “host cell” is meant a cell which contains a vector and supports the replication and/or expression of the vector. Host cells may be prokaryotic cells such as E. coli, or eukaryotic cells such as yeast, insect, amphibian, or mammalian cells.

The term “hybridization complex” includes reference to a duplex nucleic acid structure formed by two single-stranded nucleic acid sequences selectively hybridized with each other.

The term “introduced” in the context of inserting a nucleic acid into a cell, means “transfection” or “transformation” or “transduction” and includes reference to the incorporation of a nucleic acid into a eukaryotic or prokaryotic cell where the nucleic acid may be incorporated into the genome of the cell (e.g., chromosome, plasmid, plastid or mitochondrial DNA), converted into an autonomous replicon, or transiently expressed (e.g., transfected mRNA).

The term “isolated” refers to material, such as a nucleic acid or a protein, which is (1) substantially or essentially free from components that normally accompany or interact with it as found in its naturally occurring environment. The isolated material optionally comprises material not found with the material in its natural environment; or (2) if the material is in its natural environment, the material has been synthetically (non-naturally) altered by deliberate human intervention to a composition and/or placed at a location in the cell (e.g., genome or subcellular organelle) not native to a material found in that environment. The alteration to yield the synthetic material can be performed on the material within or removed from its natural state. For example, a naturally occurring nucleic acid becomes an isolated nucleic acid if it is altered, or if it is transcribed from DNA which has been altered, by means of human intervention performed within the cell from which it originates. See, e.g., Compounds and Methods for Site Directed Mutagenesis in Eukaryotic Cells, Kmiec, U.S. Pat. No. 5,565,350; In Vivo Homologous Sequence Targeting in Eukaryotic Cells; Zarling et al., PCT/US93/03868. Likewise, a naturally occurring nucleic acid (e.g., a promoter) becomes isolated if it is introduced by nonnaturally occurring means to a locus of the genome not native to that nucleic acid. Nucleic acids which are “isolated” as defined herein, are also referred to as “heterologous” nucleic acids.

Unless otherwise stated, the any reference to a specific protein encoding nucleic acid, such as I-Tev I nucleic acid, I-TevI fusion nucleic acid etc means a nucleic acid comprising a polynucleotide (an I-TevI polynucleotide, I-TevI fusion polypeptide) encoding an I-Tev polypeptide with I-TevI cleavage activity and includes all conservatively modified variants, homologs, paralogs and the like.

As used herein, “nucleic acid” includes reference to a deoxyribonucleotide or ribonucleotide polymer in either single- or double-stranded form, and unless otherwise limited, encompasses known analogues having the essential nature of natural nucleotides in that they hybridize to single-stranded nucleic acids in a manner similar to naturally occurring nucleotides (e.g., peptide nucleic acids).

As used herein, “polynucleotide” includes reference to a deoxyribopolynucleotide, ribopolynucleotide, or analogs thereof that have the essential nature of a natural ribonucleotide in that they hybridize, under stringent hybridization conditions, to substantially the same nucleotide sequence as naturally occurring nucleotides and/or allow translation into the same amino acid (s) as the naturally occurring nucleotide (s). A polynucleotide can be full-length or a subsequence of a native or heterologous structural or regulatory gene. Unless otherwise indicated, the term includes reference to the specified sequence as well as the complementary sequence thereof. Thus, DNAs or RNAs with backbones modified for stability or for other reasons are “polynucleotides” as that term is intended herein. Moreover, DNAs or RNAs comprising unusual bases, such as inosine, or modified bases, such as tritylated bases, to name just two examples, are polynucleotides as the term is used herein. It will be appreciated that a great variety of modifications have been made to DNA and RNA that serve many useful purposes known to those of skill in the art.

The term polynucleotide as it is employed herein embraces such chemically, enzymatically or metabolically modified forms of polynucleotides, as well as the chemical forms of DNA and RNA characteristic of viruses and cells, including among other things, simple and complex cells.

The terms “polypeptide”, “peptide” and “protein” are used interchangeably herein to refer to a polymer of amino acid residues. The terms apply to amino acid polymers in which one or more amino acid residue is an artificial chemical analogue of a corresponding naturally occurring amino acid, as well as to naturally occurring amino acid polymers. The essential nature of such analogues of naturally occurring amino acids is that, when incorporated into a protein, that protein is specifically reactive to antibodies elicited to the same protein but consisting entirely of naturally occurring amino acids. The terms “polypeptide”, “peptide” and “protein” are also inclusive of modifications including, but not limited to, glycosylation, lipid attachment, sulfation, gamma-carboxylation of glutamic acid residues, hydroxylation and ADP-ribosylation. It will be appreciated, as is well known and as noted above, that polypeptides are not always entirely linear. For instance, polypeptides may be branched as a result of ubiquitization, and they may be circular, with or without branching, generally as a result of posttranslation events, including natural processing event and events brought about by human manipulation which do not occur naturally. Circular, branched and branched circular polypeptides may be synthesized by non-translation natural process and by entirely synthetic methods, as well. Further, this invention contemplates the use of both the methionine-containing and the methionine-less amino terminal variants of the protein of the invention.

As used herein “promoter” includes reference to a region of DNA upstream from the start of transcription and involved in recognition and binding of RNA polymerase and other proteins to initiate transcription. Examples of promoters under developmental control include promoters that preferentially initiate transcription in certain tissues. Such promoters are referred to as “tissue preferred”. Promoters which initiate transcription only in certain tissue are referred to as “tissue specific”. A “cell type” specific promoter primarily drives expression in certain cell types in one or more organs. An “inducible” or “repressible” promoter is a promoter which is under environmental control. Tissue specific, tissue preferred, cell type specific, and inducible promoters constitute the class of “non-constitutive” promoters. A “constitutive” promoter is a promoter which is active under most environmental conditions.

As used herein “recombinant or genetically modified” includes reference to a cell or vector, that has been modified by the introduction of a heterologous nucleic acid or that the cell is derived from a cell so modified. Thus, for example, recombinant cells express genes that are not found in identical form within the native (non-recombinant) form of the cell or express native genes that are otherwise abnormally expressed, under-expressed or not expressed at all as a result of deliberate human intervention. The term “recombinant or genetically modified” as used herein does not encompass the alteration of the cell or vector by naturally occurring events (e.g., spontaneous mutation, natural transformation/transduction/transposition) such as those occurring without deliberate human intervention.

As used herein, an “expression cassette” is a nucleic acid construct, generated recombinantly or synthetically, with a series of specified nucleic acid elements which permit transcription of a particular nucleic acid in a host cell. The recombinant expression cassette can be incorporated into a plasmid, chromosome, mitochondrial DNA, plastid DNA, virus, or nucleic acid fragment. Typically, the recombinant expression cassette portion of an expression vector includes, among other sequences, a nucleic acid to be transcribed, and a promoter.

The term “residue” or “amino acid residue” or “amino acid” is used interchangeably herein to refer to an amino acid that is incorporated into a protein, polypeptide, or peptide (collectively “protein”). The amino acid may be a naturally occurring amino acid and, unless otherwise limited, may encompass non-natural analogs of natural amino acids that can function in a similar manner as naturally occurring amino acids.

The term “selectively hybridizes” includes reference to hybridization, under stringent hybridization conditions, of a nucleic acid sequence to as other biologics. Thus, under designated immunoassay conditions, the specified antibodies bind to an analyte having the recognized epitope to a substantially greater degree (e.g., at least 2-fold over background) than to substantially all analytes lacking the epitope which are present in the sample. Specific binding to an antibody under such conditions may require an antibody that is selected for its specificity for a particular protein. For example, antibodies raised to the polypeptides of the present invention can be selected from to obtain antibodies specifically reactive with polypeptides of the present invention. The proteins used as immunogens can be in native conformation or denatured so as to provide a linear epitope.

The term “stringent conditions” or “stringent hybridization conditions” includes reference to conditions under which a probe will hybridize to its target sequence, to a detectably greater degree than to other sequences (e.g., at least 2-fold over background). Stringent conditions are sequence-dependent and will be different in different circumstances. By controlling the stringency of the hybridization and/or washing conditions, target sequences can be identified which are 100% complementary to the probe (homologous probing).

Alternatively, stringency conditions can be adjusted to allow some mismatching in sequences so that lower degrees of similarity are detected (heterologous probing). Generally, a probe is less than about 1000 nucleotides in length, optionally less than 500 nucleotides in length.

Typically, stringent conditions will be those in which the salt concentration is less than about 1.5 M Na ion, typically about 0.01 to 1.0 M Na ion concentration (or other salts) at pH 7.0 to 8.3 and the temperature is at least about 30 C for short probes (e.g., 10 to 50 nucleotides) and at least about 60 C for long probes (e.g., greater than 50 nucleotides). Stringent conditions may also be achieved with the addition of destabilizing agents such as formamide. Exemplary low stringency conditions include hybridization with a buffer solution of 30 to 35% formamide, 1 MNaCl, 1% SDS (sodium dodecyl sulphate) at 37 C, and a wash in 1× to 2×SSC (20×SSC=3.0 MNaCl/0.3 M trisodium citrate) at 50 to 55 C. Exemplary moderate stringency conditions include hybridization in 40 to 45% formamide, 1 MNaCl, 1% SDS at 37 C, and a wash in <RTI 0.5× to 1×SSC at 55 to 60 C. Exemplary high stringency conditions include hybridization in 50% formamide, 1 MNaCl, 1% SDS at 37 C, and a wash in 0.1×SSC at 60 to 65 C. Specificity is typically the function of post-hybridization washes, the critical factors being the ionic strength and temperature of the final wash solution. For DNA/DNA hybrids, the Tm can be approximated from the equation of Meinkoth and Wahl, Anal. Biochem., 138: 267-284 (1984):Tm=81.5 C+16.6 (log M)+0.41 (% GC)-0.61 (% form)-500/L; where M is the molarity of monovalent cations, % GC is the percentage of guanosine and cytosine nucleotides in the DNA, % form is the percentage of formamide in the hybridization solution, and L is the length of the hybrid in base pairs. The Tm is the temperature (under defined ionic strength and pH) at which 50% of a complementary target sequence hybridizes to a perfectly matched probe. Tm is reduced by about 1 C for each 1% of mismatching; thus, Tm, hybridization and/or wash conditions can be adjusted to hybridize to sequences of the desired identity. For example, if sequences with >90% identity are sought, the Tm can be decreased 10 C. Generally, stringent conditions are selected to be about 5 C lower than the thermal melting point (Tm) for the specific sequence and its complement at a defined ionic strength and pH. However, severely stringent conditions can utilize a hybridization and/or wash at 1, 2, 3, or 4 C lower than the thermal melting point (Tm); moderately stringent conditions can utilize a hybridization and/or wash at 6, 7, 8, 9, or 10 C lower than the thermal melting point (Tm); low stringency conditions can utilize a hybridization and/or wash at 11, 12, 13, 14, 15, or 20 C lower than the thermal melting point (Tm). Using the equation, hybridization and wash compositions, and desired Tm, those of ordinary skill will understand that variations in the stringency of hybridization and/or wash solutions are inherently described. If the desired degree of mismatching results in a Tm of less than 45 C (aqueous solution) or 32 C (formamide solution) it is preferred to increase the SSC concentration so that a higher temperature can be used. An extensive guide to the hybridization of nucleic acids is found in Tijssen, Laboratory Techniques in Biochemistry and Molecular Biology—Hybridization with Nucleic Acid Probes, Part I, Chapter 2“Overview of principles of hybridization and the strategy of nucleic acid probe assays”, Elsevier, New York (1993); and Current Protocols in Molecular Biology, Chapter 2, Ausubel, et al., Eds., Greene Publishing and Wiley-Interscience, New York (1995).

As used herein, “vector” includes reference to a nucleic acid used in transfection of a host cell and into which can be inserted a polynucleotide. Vectors are often replicons. Expression vectors permit transcription of a nucleic acid inserted therein.

The following terms are used to describe the sequence relationships between a polynucleotide/polypeptide of the present invention with a reference polynucleotide/polypeptide: (a)“reference sequence”, (b)“comparison window”, (c) “sequence identity”, and (d)“percentage of sequence identity”.

(a) As used herein, “reference sequence” is a defined sequence used as a basis for sequence comparison with a polynucleotide/polypeptide of the present invention. A reference sequence may be a subset or the entirety of a specified sequence; for example, as a segment of a full-length cDNA or gene sequence, or the complete cDNA or gene sequence.

(b) As used herein, “comparison window” includes reference to a contiguous and specified segment of a polynucleotide/polypeptide sequence, wherein the polynucleotide/polypeptide sequence may be compared to a reference sequence and wherein the portion of the polynucleotide/polypeptide sequence in the comparison window may comprise additions or deletions (i.e., gaps) compared to the reference sequence (which does not comprise additions or deletions) for optimal alignment of the two sequences. Generally, the comparison window is at least 20 contiguous nucleotides/amino acids residues in length, and optionally can be 30, 40, 50, 100, or longer. Those of skill in the art understand that to avoid a high similarity to a reference sequence due to inclusion of gaps in the polynucleotide/polypeptide sequence, a gap penalty is typically introduced and is subtracted from the number of matches.

Methods of alignment of sequences for comparison are well-known in the art. Optimal alignment of sequences for comparison may be conducted by the local homology algorithm of Smith and Waterman, Adv. Appl. Math. 2: 482(1981); by the homology alignment algorithm of Needleman and Wunsch, J. Mol. Biol. 48: 443 (1970); by the search for similarity method of Pearson and Lipman, Proc. Natl. Acad. Sci. 85: 2444 (1988); by computerized implementations of these algorithms, including, but not limited to: CLUSTAL in the PC/Gene program by Intelligenetics, Mountain View, Calif.; GAP, BESTFIT, BLAST, FASTA, and TFASTA in the . . . in the GCG Wisconsin Genetics Software Package, Version 10 (available from Accelrys Inc., 9685 Scranton Road, San Diego, Calif., USA). The CLUSTAL program is well described by Higgins and Sharp, Gene 73: 237-244 (1988); Higgins and Sharp, CABIOS 5: 151-153 (1989); Corpet, et al., Nucleic Acids Research 16: 10881-90 (1988); Huang, et al., Computer Applications in the Biosciences 8: 155-65 (1992), and Pearson, et al., Methods in Molecular Biology 24: 307-331 (1994).

The BLAST family of programs which can be used for database similarity searches includes: BLASTN for nucleotide query sequences against nucleotide database sequences; BLASTX for nucleotide query sequences against protein database sequences; BLASTP for protein query sequences against protein database sequences; TBLASTN for protein query sequences against nucleotide database sequences; and TBLASTX for nucleotide query sequences against nucleotide database sequences. See, Current Protocols in Molecular Biology, Chapter 19, Ausubel, et al., Eds., Greene Publishing and Wiley-Interscience, New York (1995); Altschul et al., J. Mol. Biol., 215: 403-410 (1990); and, Altschul et al., Nucleic Acids Res. 25: 3389-3402 (1997).

Software for performing BLAST analyses is publicly available, e.g., through the National Center for Biotechnology Information www at ncbi.nlm.nih.gov/). This algorithm involves first identifying high scoring sequence pairs (HSPs) by identifying short words of length W in the query sequence, which either match or satisfy some positive-valued threshold score T when aligned with a word of the same length in a database sequence. T is referred to as the neighborhood word score threshold. These initial neighborhood word hits act as seeds for initiating searches to find longer HSPs containing them. The word hits are then extended in both directions along each sequence for as far as the cumulative alignment score can be increased. Cumulative scores are calculated using, for nucleotide sequences, the parameters M (reward score for a pair of matching residues; always >0) and N (penalty score for mismatching residues; always <0). For amino acid sequences, a scoring matrix is used to calculate the cumulative score.

Extension of the word hits in each direction are halted when: the cumulative alignment score falls off by the quantity X from its maximum achieved value; the cumulative score goes to zero or below, due to the accumulation of one or more negative-scoring residue alignments; or the end of either sequence is reached. The BLAST algorithm parameters W, T, and X determine the sensitivity and speed of the alignment. The BLASTN program (for nucleotide sequences) uses as defaults a word length (W) of 11, an expectation (E) of 10, a cutoff of 100, M=5, N=−4, and a comparison of both strands. For amino acid sequences, the BLASTP program uses as defaults a word length (W) of 3, an expectation (E) of 10, and the BLOSUM62 scoring matrix (see Henikoff & Henikoff (1989) Proc. Natl. Acad. Sci. USA 89:10915).

In addition to calculating percent sequence identity, the BLAST algorithm also performs a statistical analysis of the similarity between two sequences (see, e.g., Karlin & Altschul, Proc. Nat'l. Acad. Sci. USA 90: 5873-5877 (1993)). One measure of similarity provided by the BLAST algorithm is the smallest sum probability (P (N)), which provides an indication of the probability by which a match between two nucleotide or amino acid sequences would occur by chance. BLAST searches assume that proteins can be modeled as random sequences. However, many real proteins comprise regions of nonrandom sequences which may be homopolymeric tracts, short-period repeats, or regions enriched in one or more amino acids. Such low-complexity regions may be aligned between unrelated proteins even though other regions of the protein are entirely dissimilar. A number of low-complexity filter programs can be employed to reduce such low-complexity alignments. For example, the SEG (Wooten and Federhen, Comput. Chem., 17: 149-163 (1993)) and XNU (Claverie and States, Comput. Chem., 17: 191-201 (1993)) low-complexity filters can be employed alone or in combination.

Unless otherwise stated, nucleotide and protein identity/similarity values provided herein are calculated using GAP (GCG Version 10) under default values. GAP (Global Alignment Program) can also be used to compare a polynucleotide or polypeptide of the present invention with a reference sequence. GAP uses the algorithm of Needleman and Wunsch (J. Mol. Biol. 48: 443-453, 1970) to find the alignment of two complete sequences that maximizes the number of matches and minimizes the number of gaps. GAP considers all possible alignments and gap positions and creates the alignment with the largest number of matched bases and the fewest gaps. It allows for the provision of a gap creation penalty and a gap extension penalty in units of matched bases. GAP must make a profit of gap creation penalty number of matches for each gap it inserts. If a gap extension penalty greater than zero is chosen, GAP must, in addition, make a profit for each gap inserted of the length of the gap times the gap extension penalty. Default gap creation penalty values and gap extension penalty values in Version 10 of the Wisconsin Genetics Software Package for protein sequences are 8 and 2, respectively. For nucleotide sequences the default gap creation penalty is 50 while the default gap extension penalty is 3. The gap creation and gap extension penalties can be expressed as an integer selected from the group of integers consisting of from 0 to 100. Thus, for example, the gap creation and gap extension penalties can each independently be: 3, 4, 5, 6, 7, 8, 9, 10, 15, 20, 30, 40, 50, 60 or greater.

GAP presents one member of the family of best alignments. There may be many members of this family, but no other member has a better quality. GAP displays four figures of merit for alignments: Quality, Ratio, Identity, and Similarity. The Quality is the metric maximized in order to align the sequences. Ratio is the quality divided by the number of bases in the shorter segment. Percent Identity is the percent of the symbols that actually match. Percent Similarity is the percent of the symbols that are similar. Symbols that are across from gaps are ignored. A similarity is scored when the scoring matrix value for a pair of symbols is greater than or equal to 0.50, the similarity threshold. The scoring matrix used in Version 10 of the Wisconsin Genetics Software Package is BLOSUM62 (see Henikoff& Henikoff (1989) Proc. Natl. Acad. Sci. USA 89: 10915).

Multiple alignment of the sequences can be performed using the CLUSTAL method of alignment (Higgins and Sharp (1989) CABIOS. 5: 151-153) with the default parameters (GAPPENALTY=10, GAP LENGTH PENALTY=10). Default parameters for pairwise alignments using the CLUSTAL method are KTUPLE 1, GAP PENALTY=3, WINDOW=5 and DIAGONALS SAVED=5.

(c) As used herein, “sequence identity” or “identity” in the context of two nucleic acid or polypeptide sequences includes reference to the residues in the two sequences which are the same when aligned for maximum correspondence over a specified comparison window. When percentage of sequence identity is used in reference to proteins it is recognized that residue positions which are not identical often differ by conservative amino acid substitutions, where amino acid residues are substituted for other amino acid residues with similar chemical properties (e.g. charge or hydrophobicity) and therefore do not change the functional properties of the molecule. Where sequences differ in conservative substitutions, the percent sequence identity may be adjusted upwards to correct for the conservative nature of the substitution. Sequences which differ by such conservative substitutions are said to have “sequence similarity” or “similarity”. Means for making this adjustment are well-known to those of skill in the art. Typically this involves scoring a conservative substitution as a partial rather than a full mismatch, thereby increasing the percentage sequence identity. Thus, for example, where an identical amino acid is given a score of 1 and a non-conservative substitution is given a score of zero, a conservative substitution is given a score between zero and 1. The scoring of conservative substitutions is calculated, e.g., according to the algorithm of Meyers and Miller, Computer Applic. Biol. Sci., 4: 11-17 (1988) e. <RTI g., as implemented in the program PC/GENE (Intelligenetics, Mountain View, Calif., USA).

(d) As used herein, “percentage of sequence identity” means the value determined by comparing two optimally aligned sequences over a comparison window, wherein the portion of the polynucleotide sequence in the comparison window may comprise additions or deletions (i.e., gaps) as compared to the reference sequence (which does not comprise additions or deletions) for optimal alignment of the two sequences. The percentage is calculated by determining the number of positions at which the identical nucleic acid base or amino acid residue occurs in both sequences to yield the number of matched positions, dividing the number of matched positions by the total number of positions in the window of comparison and multiplying the result by 100 to yield the percentage of sequence identity.

### Targeted Cleavage of DNA

### Target Sites

The disclosed methods and compositions include fusion proteins comprising a cleavage domain derived from I-TevI fused to either a TAL effector DNA binding domain, or DNA recognition sequence in which the RVDs, by binding to a sequence in cellular chromatin (e.g., a target site or a binding site), directs the activity of the cleavage domain (or cleavage half-domain) to the vicinity of the sequence and, hence, induces cleavage in the vicinity of the target sequence, or a zinc finger protein motel. As set forth elsewhere in this disclosure, particular RVDs within a TAL binding domain or zinc finger domain can be engineered to bind to virtually any desired sequence. Accordingly, after identifying a region of interest containing a sequence at which cleavage or recombination is desired, one or more TAL effector, or zinc finger DNA binding domains can be engineered to bind to one or more sequences in the region of interest. Expression of a fusion protein comprising a TAL effector or zinc finger DNA binding domain and an I-Tev-I cleavage domain, in a cell, effects cleavage in the region of interest.

Selection of a sequence in cellular chromatin for binding by a TAL effector of zinc finger binding domain (e.g., a target site) can be accomplished, by any method known to those of skill in the art. For example simple visual inspection of a nucleotide sequence can be used for selection of a target site. Accordingly, any means for target site selection can be used in the claimed methods.

### Sequence-Specific Endonucleases

Sequence-specific nucleases and recombinant nucleic acids encoding the sequence-specific endonucleases are provided herein. The sequence-specific endonucleases can any binding domain such as a TAL effector DNA binding domains or zinc finger binding domain and endonuclease domains. Thus, nucleic acids encoding such sequence-specific endonucleases can include a nucleotide sequence from a sequence-specific TAL effector or zinc finger linked to a nucleotide sequence from a nuclease, such as the novel nuclease of the invention.

TAL Effector

TAL effectors are proteins of plant pathogenic bacteria that are injected by the pathogen into the plant cell, where they travel to the nucleus and function as transcription factors to turn on specific plant genes. The primary amino acid sequence of a TAL effector dictates the nucleotide sequence to which it binds. Because the relationship between the TAL amino acid sequence and the target binding site is simple, target sites can be predicted for TAL effectors, and TAL effectors also can be engineered and generated for the purpose of binding to particular nucleotide sequences.

### Zinc Finger Binding Domain

Zinc finger binding domains may be engineered to recognize and bind to any nucleic acid sequence of choice. See, for example, Beerli et al. (2002) Nature Biotechnol. 20:135-141; Pabo et al. (2001) Ann. Rev. Biochem. 70:313-340; Isalan et al. (2001) Nature Biotechnol. 19:656-660; Segal et al. (2001) Curr. Opin. Biotechnol. 12:632-637; and Choo et al. (2000) Curr. Opin. Struct. Biol. 10:411-416. An engineered zinc finger binding domain may have a novel binding specificity compared to a naturally-occurring zinc finger protein. Engineering methods include, but are not limited to, rational design and various types of selection. Rational design includes, for example, using databases comprising doublet, triplet, and/or quadruplet nucleotide sequences and individual zinc finger amino acid sequences, in which each doublet, triplet or quadruplet nucleotide sequence is associated with one or more amino acid sequences of zinc fingers which bind the particular triplet or quadruplet sequence. See, for example, U.S. Pat. Nos. 6,453,242 and 6,534,261, the disclosures of which are incorporated by reference herein in their entireties. As an example, the algorithm of described in U.S. Pat. No. 6,453,242 may be used to design a zinc finger binding domain to target a preselected sequence. Alternative methods, such as rational design using a nondegenerate recognition code table may also be used to design a zinc finger binding domain to target a specific sequence (see, for example, Biochemistry 2002, 41, 7074-7081).

A zinc finger binding domain may be designed to recognize a DNA sequence ranging from about 3 nucleotides to about 21 nucleotides in length, or from about 8 to about 19 nucleotides in length. In general, the zinc finger binding domains of the zinc finger nucleases disclosed herein comprise at least three zinc finger recognition regions (i.e., zinc fingers). In one embodiment, the zinc finger binding domain may comprise four zinc finger recognition regions. In another embodiment, the zinc finger binding domain may comprise five zinc finger recognition regions. In still another embodiment, the zinc finger binding domain may comprise six zinc finger recognition regions. A zinc finger binding domain may be designed to bind to any suitable target DNA sequence. See for example, U.S. Pat. Nos. 6,607,882; 6,534,261 and 6,453,242, the disclosures of which are incorporated by reference herein in their entireties.

Exemplary methods of selecting a zinc finger recognition region may include phage display and two-hybrid systems, and are disclosed in U.S. Pat. Nos. 5,789,538; 5,925,523; 6,007,988; 6,013,453; 6,410,248; 6,140,466; 6,200,759; and 6,242,568; as well as WO 98/37186; WO 98/53057; WO 00/27878; WO 01/88197 and GB 2,338,237, each of which is incorporated by reference herein in its entirety. In addition, enhancement of binding specificity for zinc finger binding domains has been described, for example, in WO 02/077227.

Zinc finger binding domains and methods for design and construction of fusion proteins (and polynucleotides encoding same) are known to those of skill in the art and are described in detail in U.S. Patent Application Publication Nos. 20050064474 and 20060188987, each incorporated by reference herein in its entirety. Zinc finger recognition regions and/or multi-fingered zinc finger proteins may be linked together using suitable linker sequences, including for example, linkers of five or more amino acids in length. See, U.S. Pat. Nos. 6,479,626; 6,903,185; and 7,153,949, the disclosures of which are incorporated by reference herein in their entireties, for non-limiting examples of linker sequences of six or more amino acids in length. The zinc finger binding domain described herein may include a combination of suitable linkers between the individual zinc fingers of the protein.

In some embodiments, the zinc finger nuclease may further comprise a nuclear localization signal or sequence (NLS). A NLS is an amino acid sequence that facilitates targeting the zinc finger nuclease protein into the nucleus to introduce a double stranded break at the target sequence in the chromosome. Nuclear localization signals are known in the art. See, for example, Makkerh et al. (1996) Current Biology 6:1025-1027.

Fused to the TAL effector-encoding nucleic acid sequences or zinc finger encoding nucleic acid sequences are sequences encoding a nuclease or a portion of a nuclease, herein, the homing nuclease I-TevI. The I-Tev I nuclease can form a functional enzyme as a monomer and as such provides a large advantage in design and targeting of sequences. It also expected that other closely related homing endonucleases, particularly of the GIY-YIG family will also function in the methods of the invention, such as, for example I-BmoI.

Homing endonucleases are grouped into at least four different families that are defined on the basis of conserved sequence elements as the GIY-YIG, LAGLIDADG, H-N-H and His-Cys box families. They recognize long DNA targets of 14-40 bp, with some degree of sequence tolerance.

GIY-YIG family members contain up to five conserved sequence motifs that make up the GIY-YIG module with essentially no similarity among the proteins beyond that. In addition to the GIY-(9 to 10 residue)-YIG sequence, this module includes several other highly conserved residues, some of which have been shown to be critical for catalytic activity. These include Tyr1 Arg27, Glu75, and Asn90 (I-TevI sequence numbers).

I-TevI specifically recognizes its 37-bp DNA substrate, or homing site, as a monomer. The primary binding region of the enzyme is approximately 20 bp in length, spanning the intron insertion site (IS), with a second region of contact close to the cleavage site (CS), which is 23-25 bp upstream of the IS. In addition, I-TevI can tolerate insertions or deletions between the CS and IS, and still effect cleavage.

A sequence-specific TAL effector or zinc finger endonuclease as provided herein can recognize a particular sequence within a preselected target nucleotide sequence present in a cell. Thus, in some embodiments, a target nucleotide sequence can be scanned for nuclease recognition sites, and a particular nuclease can be selected based on the target sequence. In other cases, a TAL effector or zinc finger endonuclease can be engineered to target a particular cellular sequence. A nucleotide sequence encoding the desired TAL effector or zinc finger endonuclease can be inserted into any suitable expression vector, and can be linked to one or more expression control sequences. For example, a nuclease coding sequence can be operably linked to a promoter sequence that will lead to constitutive expression of the endonuclease in the species of plant to be transformed. Alternatively, an endonuclease coding sequence can be operably linked to a promoter sequence that will lead to conditional expression (e.g., expression under certain nutritional conditions).

### TAL Effector DNA Domain-Cleavage Domain Fusions

Methods for design and construction of fusion proteins (and polynucleotides encoding same) are known to those of skill in the art. For example, methods for the design and construction of fusion protein comprising TAL proteins (and polynucleotides encoding same) are described in U.S. Pat. Nos. 6,453,242 and 6,534,261. In certain embodiments, polynucleotides encoding such fusion proteins are constructed. These polynucleotides can be inserted into a vector and the vector can be introduced into a cell (see below for additional disclosure regarding vectors and methods for introducing polynucleotides into cells).

In certain embodiments of the methods described herein, a fusion protein comprises a TAL effector binding domain from AvrXa7 and a cleavage domain from the I-TevI homing endonuclease the monomer fusion protein is then expressed in a cell. Expression of the fusion protein in a cell can result from delivery of the protein to the cell; a nucleic acid encoding the protein to the cell.

In certain embodiments, the components of the fusion proteins (e.g, TAL-I-TevI fusions) are arranged such that the cleavage domain is nearest the amino terminus of the fusion protein, and the TAL domain is nearest the carboxy-terminus. This provides certain advantages such as the retention of the transcription activator activity which enables one to measure the DNA binding specificity of naturally occurring TAL or newly engineered TAL used for nuclease fusion and this orientation may give the flexibility of spacer lengths.

### Zinc Finger DNA Domain-Cleavage Domain Fusions

Methods for design and construction of fusion proteins (and polynucleotides encoding same) are known to those of skill in the art. For example, methods for the design and construction of fusion protein comprising zinc finger binding domains (and polynucleotides encoding same) are described in U.S. Pat. Nos. 5,789,538; 5,925,523; 6,007,988; 6,013,453; 6,410,248; 6,140,466; 6,200,759; and 6,242,568; as well as WO 98/37186; WO 98/53057; WO 00/27878; WO 01/88197 and GB 2,338,237. In certain embodiments, polynucleotides encoding such fusion proteins are constructed. These polynucleotides can be inserted into a vector and the vector can be introduced into a cell (see below for additional disclosure regarding vectors and methods for introducing polynucleotides into cells).

Expression of the fusion protein in a cell can result from delivery of the protein to the cell; a nucleic acid encoding the protein to the cell.

### Methods for Targeted Cleavage

The disclosed methods and compositions can be used to cleave DNA at a region of interest in cellular chromatin (e.g., at a desired or predetermined site in a genome, for example, in a gene, either mutant or wild-type). For such targeted DNA cleavage, a binding domain such as TAL or zinc finger, is engineered to bind a target site at or near the predetermined cleavage site, and a fusion protein comprising the engineered binding domain and a I-TevI cleavage domain is expressed in a cell. Upon binding of the binding portion of the fusion protein to the target site, the DNA is cleaved near the target site by the cleavage domain.

For targeted cleavage using a binding domain-cleavage domain fusion polypeptide, the binding site can encompass the cleavage site, or the near edge of the binding site can be 1, 2, 3, 4, 5, 6, 10, 25, 50 or more nucleotides (or any integral value between 1 and 50 nucleotides) from the cleavage site. The exact location of the binding site, with respect to the cleavage site, will depend upon the particular cleavage domain, and the length of any linker.

Thus, the methods described herein can employ an engineered TAL effector DNA binding domain or a zinc finger binding domain fused to the I-TevI cleavage domain of the invention. In these cases, the binding domain is engineered to bind to a target sequence, at or near which cleavage is desired. The fusion protein, or a polynucleotide encoding same, is introduced into a cell. Once introduced into, or expressed in, the cell, the fusion protein binds to the target sequence and cleaves at or near the target sequence. The exact site of cleavage depends on the nature of the cleavage domain and/or the presence and/or nature of linker sequences between the binding and cleavage domains. Optimal levels of cleavage can also depend on both the distance between the binding sites of the two fusion proteins (See, for example, Smith et al. (2000) Nucleic Acids Res. 28:3361-3369; Bibikova et al. (2001) Mol. Cell. Biol. 21:289-297) and the length of the ZC linker in each fusion protein.

The site at which the DNA is cleaved generally lies between the binding sites for the two fusion proteins. Double-strand breakage of DNA often results from two single-strand breaks, or “nicks,” offset by 1, 2, 3, 4, 5, 6 or more nucleotides.

As noted above, the fusion protein(s) can be introduced as polypeptides and/or polynucleotides. For example, two polynucleotides, each comprising sequences encoding one of the aforementioned polypeptides, can be introduced into a cell, and when the polypeptides are expressed and each binds to its target sequence, cleavage occurs at or near the target sequence. Alternatively, a single polynucleotide comprising sequences encoding both fusion polypeptides is introduced into a cell. Polynucleotides can be DNA, RNA or any modified forms or analogues or DNA and/or RNA.

To enhance cleavage specificity, additional compositions may also be employed in the methods described herein. For example, single cleavage domains can exhibit limited double-stranded cleavage activity.

In addition to the fusion molecules described herein, targeted replacement of a selected genomic sequence also requires the introduction of the replacement (or donor) sequence. The donor sequence can be introduced into the cell prior to, concurrently with, or subsequent to, expression of the fusion protein(s). The donor polynucleotide contains sufficient homology to a genomic sequence to support homologous recombination between it and the genomic sequence to which it bears homology. Approximately 25, 50, 100 or 200 nucleotides or more of sequence homology between a donor and a genomic sequence (or any integral value between 10 and 200 nucleotides, or more) will support homologous recombination therebetween. Donor sequences can range in length from 10 to 5,000 nucleotides (or any integral value of nucleotides therebetween) or longer. It will be readily apparent that the donor sequence is typically not identical to the genomic sequence that it replaces. For example, the sequence of the donor polynucleotide can contain one or more single base changes, insertions, deletions, inversions or rearrangements with respect to the genomic sequence, so long as sufficient homology is present to support homologous recombination. Alternatively, a donor sequence can contain a non-homologous sequence flanked by two regions of homology. Additionally, donor sequences can comprise a vector molecule containing sequences that are not homologous to the region of interest in cellular chromatin. Generally, the homologous region(s) of a donor sequence will have at least 50% sequence identity to a genomic sequence with which recombination is desired. In certain embodiments, 60%, 70%, 80%, 90%, 95%, 98%, 99%, or 99.9% sequence identity is present. Any value between 1% and 100% sequence identity can be present, depending upon the length of the donor polynucleotide.

A donor molecule can contain several, discontinuous regions of homology to cellular chromatin. For example, for targeted insertion of sequences not normally present in a region of interest, said sequences can be present in a donor nucleic acid molecule and flanked by regions of homology to sequence in the region of interest.

To simplify assays (e.g., hybridization, PCR, restriction enzyme digestion) for determining successful insertion of the donor sequence, certain sequence differences may be present in the donor sequence as compared to the genomic sequence. Preferably, if located in a coding region, such nucleotide sequence differences will not change the amino acid sequence, or will make silent amino acid changes (i.e., changes which do not affect the structure or function of the protein). The donor polynucleotide can optionally contain changes in sequences corresponding to the TAL effector domain binding (or recognition) sites in the region of interest, to prevent cleavage of donor sequences that have been introduced into cellular chromatin by homologous recombination.

The donor polynucleotide can be DNA or RNA, single-stranded or double-stranded and can be introduced into a cell in linear or circular form. If introduced in linear form, the ends of the donor sequence can be protected (e.g., from exonucleolytic degradation) by methods known to those of skill in the art. For example, one or more dideoxynucleotide residues are added to the 3′ terminus of a linear molecule and/or self-complementary oligonucleotides are ligated to one or both ends. See, for example, Chang et al. (1987) Proc. Natl. Acad. Sci. USA 84:4959-4963; Nehls et al. (1996) Science 272:886-889. Additional methods for protecting exogenous polynucleotides from degradation include, but are not limited to, addition of terminal amino group(s) and the use of modified internucleotide linkages such as, for example, phosphorothioates, phosphoramidates, and O-methyl ribose or deoxyribose residues. A polynucleotide can be introduced into a cell as part of a vector molecule having additional sequences such as, for example, replication origins, promoters and genes encoding antibiotic resistance. Moreover, donor polynucleotides can be introduced as naked nucleic acid, as nucleic acid complexed with an agent such as a liposome or poloxamer, or can be delivered by viruses (e.g., adenovirus, AAV).

Without being bound by one theory, it appears that the presence of a double-stranded break in a cellular sequence, coupled with the presence of an exogenous DNA molecule having homology to a region adjacent to or surrounding the break, activates cellular mechanisms which repair the break by transfer of sequence information from the donor molecule into the cellular (e.g., genomic or chromosomal) sequence; i.e., by a processes of homologous recombination. Applicants' methods advantageously combine the powerful targeting capabilities of engineered TALs with a cleavage domain (or cleavage half-domain) to specifically target a double-stranded break to the region of the genome at which recombination is desired.

For alteration of a chromosomal sequence, it is not necessary for the entire sequence of the donor to be copied into the chromosome, as long as enough of the donor sequence is copied to effect the desired sequence alteration.

In certain embodiments, a homologous chromosome can serve as the donor polynucleotide. Thus, for example, correction of a mutation in a heterozygote can be achieved by engineering fusion proteins which bind to and cleave the mutant sequence on one chromosome, but do not cleave the wild-type sequence on the homologous chromosome. The double-stranded break on the mutation-bearing chromosome stimulates a homology-based “gene conversion” process in which the wild-type sequence from the homologous chromosome is copied into the cleaved chromosome, thus restoring two copies of the wild-type sequence.

Further increases in efficiency of targeted recombination, in cells comprising fusion molecule and a donor DNA molecule, are achieved by blocking the cells in the G₂ phase of the cell cycle, when homology-driven repair processes are maximally active. Such arrest can be achieved in a number of ways. For example, cells can be treated with e.g., drugs, compounds and/or small molecules which influence cell-cycle progression so as to arrest cells in G₂ phase. Exemplary molecules of this type include, but are not limited to, compounds which affect microtubule polymerization (e.g., vinblastine, nocodazole, Taxol), compounds that interact with DNA (e.g., cis-platinum(II) diamine dichloride, Cisplatin, doxorubicin) and/or compounds that affect DNA synthesis (e.g., thymidine, hydroxyurea, L-mimosine, etoposide, 5-fluorouracil). Additional increases in recombination efficiency are achieved by the use of histone deacetylase (HDAC) inhibitors (e.g., sodium butyrate, trichostatin A) which alter chromatin structure to make genomic DNA more accessible to the cellular recombination machinery.

Additional methods for cell-cycle arrest include overexpression of proteins which inhibit the activity of the CDK cell-cycle kinases, for example, by introducing a cDNA encoding the protein into the cell or by introducing into the cell an engineered ZFP which activates expression of the gene encoding the protein. Cell-cycle arrest is also achieved by inhibiting the activity of cyclins and CDKs, for example, using RNAi methods (e.g., U.S. Pat. No. 6,506,559) or by introducing into the cell an engineered ZFP which represses expression of one or more genes involved in cell-cycle progression such as, for example, cyclin and/or CDK genes. See, e.g., U.S. Pat. No. 6,534,261 for methods for the synthesis of engineered TAL proteins for regulation of gene expression.

Methods to Screen for Cellular Factors that Facilitate Homologous Recombination

Since homologous recombination is a multi-step process requiring the modification of DNA ends and the recruitment of several cellular factors into a protein complex, the addition of one or more exogenous factors, along with donor DNA and vectors encoding binding domain-cleavage domain fusions, can be used to facilitate targeted homologous recombination. An exemplary method for identifying such a factor or factors employs analyses of gene expression using microarrays (e.g., Affymetrix Gene Chip® arrays) to compare the mRNA expression patterns of different cells. For example, cells that exhibit a higher capacity to stimulate double strand break-driven homologous recombination in the presence of donor DNA and binding domain-cleavage domain fusions, either unaided or under conditions known to increase the level of gene correction, can be analyzed for their gene expression patterns compared to cells that lack such capacity. Genes that are upregulated or downregulated in a manner that directly correlates with increased levels of homologous recombination are thereby identified and can be cloned into any one of a number of expression vectors. These expression constructs can be co-transfected along with binding domain-cleavage domain fusions and donor constructs to yield improved methods for achieving high-efficiency homologous recombination.

### Expression Vectors

A nucleic acid encoding one or more fusion proteins can be cloned into a vector for transformation into prokaryotic or eukaryotic cells for replication and/or expression. Vectors can be prokaryotic vectors, e.g., plasmids, or shuttle vectors, insect vectors, or eukaryotic vectors. A nucleic acid encoding a TAL effector binding domain or zinc finger domain can also be cloned into an expression vector, for administration to a plant cell, animal cell, preferably a mammalian cell or a human cell, fungal cell, bacterial cell, or protozoal cell.

To obtain expression of a cloned gene or nucleic acid, sequences encoding a fusion protein are typically subcloned into an expression vector that contains a promoter to direct transcription.

Promoters are involved in recognition and binding of RNA polymerase and other proteins to initiate and modulate transcription. To bring a coding sequence under the control of a promoter, it typically is necessary to position the translation initiation site of the translational reading frame of the polypeptide between one and about fifty nucleotides downstream of the promoter. A promoter can, however, be positioned as much as about 5,000 nucleotides upstream of the translation start site, or about 2,000 nucleotides upstream of the transcription start site. A promoter typically comprises at least a core (basal) promoter. A promoter also may include at least one control element such as an upstream element. Such elements include upstream activation regions (UARs) and, optionally, other DNA sequences that affect transcription of a polynucleotide such as a synthetic upstream element.

The choice of promoters to be included depends upon several factors, including, but not limited to, efficiency, selectability, inducibility, desired expression level, and cell or tissue specificity. For example, tissue-, organ- and cell-specific promoters that confer transcription only or predominantly in a particular tissue, organ, and cell type, respectively, can be used. In some embodiments, promoters specific to vegetative tissues such as the stem, parenchyma, ground meristem, vascular bundle, cambium, phloem, cortex, shoot apical meristem, lateral shoot meristem, root apical meristem, lateral root meristem, leaf primordium, leaf mesophyll, or leaf epidermis can be suitable regulatory regions. In some embodiments, promoters that are essentially specific to seeds (“seed-preferential promoters”) can be useful. Seed-specific promoters can promote transcription of an operably linked nucleic acid in endosperm and cotyledon tissue during seed development. Alternatively, constitutive promoters can promote transcription of an operably linked nucleic acid in most or all tissues of a plant, throughout plant development. Other classes of promoters include, but are not limited to, inducible promoters, such as promoters that confer transcription in response to external stimuli such as chemical agents, developmental stimuli, or environmental stimuli.

A basal promoter is the minimal sequence necessary for assembly of a transcription complex required for transcription initiation. Basal promoters frequently include a “TATA box” element that may be located between about 15 and about 35 nucleotides upstream from the site of transcription initiation. Basal promoters also may include a “CCAAT box” element (typically the sequence CCAAT) and/or a GGGCG sequence, which can be located between about 40 and about 200 nucleotides, typically about 60 to about 120 nucleotides, upstream from the transcription start site.

Non-limiting examples of promoters that can be included in the nucleic acid constructs provided herein include the cauliflower mosaic virus (CaMV) 35S transcription initiation region, the 1′ or 2′ promoters derived from T-DNA of Agrobacterium tumefaciens, promoters from a maize leaf-specific gene described by Busk ((1997) Plant J 11:1285-1295), kn1-related genes from maize and other species, and transcription initiation regions from various plant genes such as the maize ubiquitin-1 promoter.

A 5′ untranslated region (UTR) is transcribed, but is not translated, and lies between the start site of the transcript and the translation initiation codon and may include the +1 nucleotide. A 3′ UTR can be positioned between the translation termination codon and the end of the transcript. UTRs can have particular functions such as increasing mRNA message stability or translation attenuation. Examples of 3′ UTRs include, but are not limited to polyadenylation signals and transcription termination sequences. A polyadenylation region at the 3′-end of a coding region can also be operably linked to a coding sequence. The polyadenylation region can be derived from the natural gene, from various other plant genes, or from an Agrobacterium T-DNA.

The vectors provided herein also can include, for example, origins of replication, and/or scaffold attachment regions (SARs). In addition, an expression vector can include a tag sequence designed to facilitate manipulation or detection (e.g., purification or localization) of the expressed polypeptide. Tag sequences, such as green fluorescent protein (GFP), glutathione S-transferase (GST), polyhistidine, c-myc, hemagglutinin, or Flag” tag (Kodak, New Haven, Conn.) sequences typically are expressed as a fusion with the encoded polypeptide. Such tags can be inserted anywhere within the polypeptide, including at either the carboxyl or amino terminus.

It will be understood that more than one regulatory region may be present in a recombinant polynucleotide, e.g., introns, enhancers, upstream activation regions, and inducible elements.

Recombinant nucleic acid constructs can include a polynucleotide sequence inserted into a vector suitable for transformation of cells (e.g., plant cells or animal cells). Recombinant vectors can be made using, for example, standard recombinant DNA techniques (see, e.g., Sambrook et al. (1989) Molecular Cloning: A Laboratory Manual, 2nd ed., Cold Spring Harbor Laboratory, Cold Spring Harbor, N.Y.).

Suitable bacterial and eukaryotic promoters are well known in the art and described, e.g., in Sambrook et al., Molecular Cloning, A Laboratory Manual (2nd ed. 1989; 3rd ed., 2001); Kriegler, Gene Transfer and Expression: A Laboratory Manual (1990); and Current Protocols in Molecular Biology (Ausubel et al., supra. Bacterial expression systems for expressing the ZFP are available in, e.g., E. coli, Bacillus sp., and Salmonella (Palva et al., Gene 22:229-235 (1983)). Kits for such expression systems are commercially available. Eukaryotic expression systems for mammalian cells, yeast, and insect cells are well known by those of skill in the art and are also commercially available.

The promoter used to direct expression of a fusion protein-encoding nucleic acid depends on the particular application. For example, a strong constitutive promoter is typically used for expression and purification of TAL or ZFN-cleavage domain fusion proteins. In contrast, when a TAL or ZFN-cleavage domain fusion protein is administered in vivo for gene regulation, either a constitutive or an inducible promoter is used, depending on the particular use of the TAL-cleavage or ZFN-cleavage domain fusion protein. In addition, a preferred promoter for administration of a TAL-cleavage or ZFN-cleavage domain fusion protein can be a weak promoter, such as HSV TK or a promoter having similar activity. The promoter typically can also include elements that are responsive to transactivation, e.g., hypoxia response elements, Gal4 response elements, lac repressor response element, and small molecule control systems such as tet-regulated systems and the RU-486 system (see, e.g., Gossen & Bujard, PNAS 89:5547 (1992); Oligino et al., Gene Ther. 5:491-496 (1998); Wang et al., Gene Ther. 4:432-441 (1997); Neering et al., Blood 88:1147-1155 (1996); and Rendahl et al., Nat. Biotechnol. 16:757-761 (1998)). The MNDU3 promoter can also be used, and is preferentially active in CD34+ hematopoietic stem cells.

In addition to the promoter, the expression vector typically contains a transcription unit or expression cassette that contains all the additional elements required for the expression of the nucleic acid in host cells, either prokaryotic or eukaryotic. A typical expression cassette thus contains a promoter operably linked, e.g., to a nucleic acid sequence encoding the TAL-cleavage or ZFN-cleavage domain fusion protein and signals required, e.g., for efficient polyadenylation of the transcript, transcriptional termination, ribosome binding sites, or translation termination. Additional elements of the cassette may include, e.g., enhancers, and heterologous splicing signals.

The particular expression vector used to transport the genetic information into the cell is selected with regard to the intended use of the TAL-cleavage or ZFN-cleavage domain fusion protein, e.g., expression in plants, animals, bacteria, fungus, protozoa, etc. (see expression vectors described below). Standard bacterial expression vectors include plasmids such as pBR322-based plasmids, pSKF, pET23D, and commercially available fusion expression systems such as GST and LacZ. An exemplary fusion protein is the maltose binding protein, “MBP.” Such fusion proteins are used for purification of the TAL-cleavage domain fusion protein. Epitope tags can also be added to recombinant proteins to provide convenient methods of isolation, for monitoring expression, and for monitoring cellular and subcellular localization, e.g., c-myc or FLAG.

Expression vectors containing regulatory elements from eukaryotic viruses are often used in eukaryotic expression vectors, e.g., SV40 vectors, papilloma virus vectors, and vectors derived from Epstein-Barr virus. Other exemplary eukaryotic vectors include pMSG, pAV009/A+, pMTO 10/A+, pMAMneo-5, baculovirus pDSVE, and any other vector allowing expression of proteins under the direction of the SV40 early promoter, SV40 late promoter, metallothionein promoter, murine mammary tumor virus promoter, Rous sarcoma virus promoter, polyhedrin promoter, or other promoters shown effective for expression in eukaryotic cells.

Some expression systems have markers for selection of stably transfected cell lines such as thymidine kinase, hygromycin B phosphotransferase, and dihydrofolate reductase. High yield expression systems are also suitable, such as using a baculovirus vector in insect cells, with a TAL-cleavage domain fusion protein encoding sequence under the direction of the polyhedrin promoter or other strong baculovirus promoters.

The elements that are typically included in expression vectors also include a replicon that functions in E. coli, a gene encoding antibiotic resistance to permit selection of bacteria that harbor recombinant plasmids, and unique restriction sites in nonessential regions of the plasmid to allow insertion of recombinant sequences.

Standard transfection methods are used to produce plant, bacterial, mammalian, yeast or insect cell lines that express large quantities of protein, which are then purified using standard techniques (see, e.g., Colley et al., J. Biol. Chem. 264:17619-17622 (1989); Guide to Protein Purification, in Methods in Enzymology, vol. 182 (Deutscher, ed., 1990)). Transformation of eukaryotic and prokaryotic cells are performed according to standard techniques (see, e.g., Morrison, J. Bact. 132:349-351 (1977); Clark-Curtiss & Curtiss, Methods in Enzymology 101:347-362 (Wu et al., eds, 1983).

Any of the well known procedures for introducing foreign nucleotide sequences into host cells may be used. These include the use of calcium phosphate transfection, polybrene, protoplast fusion, electroporation, ultrasonic methods (e.g., sonoporation), liposomes, microinjection, naked DNA, plasmid vectors, viral vectors, both episomal and integrative, and any of the other well known methods for introducing cloned genomic DNA, cDNA, synthetic DNA or other foreign genetic material into a host cell (see, e.g., Sambrook et al., supra). It is only necessary that the particular genetic engineering procedure used be capable of successfully introducing at least one gene into the host cell capable of expressing the protein of choice.

### Nucleic Acids Encoding Fusion Proteins and Delivery to Cells

Conventional viral and non-viral based gene transfer methods can be used to introduce nucleic acids encoding engineered TAL-cleavage domain fusion proteins in animal cells (e.g., mammalian cells) and target tissues. Such methods can also be used to administer nucleic acids encoding TAL-cleavage domain fusion proteins to cells in vitro. In certain embodiments, nucleic acids encoding TAL-cleavage domain fusion proteins are administered for in vivo or ex vivo gene therapy uses. Non-viral vector delivery systems include DNA plasmids, naked nucleic acid, and nucleic acid complexed with a delivery vehicle such as a liposome or poloxamer. Viral vector delivery systems include DNA and RNA viruses, which have either episomal or integrated genomes after delivery to the cell. For a review of gene therapy procedures, see Anderson, Science 256:808-813 (1992); Nabel & Felgner, TIBTECH 11:211-217 (1993); Mitani & Caskey, TIBTECH 11:162-166 (1993); Dillon, TIBTECH 11:167-175 (1993); Miller, Nature 357:455-460 (1992); Van Brunt, Biotechnology 6(10): 1149-1154 (1988); Vigne, Restorative Neurology and Neuroscience 8:35-36 (1995); Kremer & Perricaudet, British Medical Bulletin 51(1):31-44 (1995); Haddada et al., in Current Topics in Microbiology and Immunology Doerfler and Bohm (eds) (1995); and Yu et al., Gene Therapy 1: 13-26 (1994).

Methods of non-viral delivery of nucleic acids encoding engineered TAL-cleavage domain fusion proteins include electroporation, lipofection, microinjection, biolistics, virosomes, liposomes, immunoliposomes, polycation or lipid:nucleic acid conjugates, naked DNA, artificial virions, and agent-enhanced uptake of DNA. Sonoporation using, e.g., the Sonitron 2000 system (Rich-Mar) can also be used for delivery of nucleic acids.

Additional exemplary nucleic acid delivery systems include those provided by Amaxa Biosystems (Cologne, Germany), Maxcyte, Inc. (Rockville, Md.) and BTX Molecular Delivery Systems (Holliston, Mass.).

The use of RNA or DNA viral based systems for the delivery of nucleic acids encoding engineered TAL-cleavage domain fusion proteins take advantage of highly evolved processes for targeting a virus to specific cells in the body and trafficking the viral payload to the nucleus. Viral vectors can be administered directly to patients (in vivo) or they can be used to treat cells in vitro and the modified cells are administered to patients (ex vivo). Conventional viral based systems for the delivery of TAL-cleavage domain fusion proteins include, but are not limited to, retroviral, lentivirus, adenoviral, adeno-associated, vaccinia and herpes simplex virus vectors for gene transfer. Integration in the host genome is possible with the retrovirus, lentivirus, and adeno-associated virus gene transfer methods, often resulting in long term expression of the inserted transgene. Additionally, high transduction efficiencies have been observed in many different cell types and target tissues.

In applications in which transient expression of a TAL-cleavage or ZFN-cleavage domain fusion protein fusion protein is preferred, adenoviral based systems can be used. Adenoviral based vectors are capable of very high transduction efficiency in many cell types and do not require cell division. With such vectors, high titer and high levels of expression have been obtained. This vector can be produced in large quantities in a relatively simple system. Adeno-associated virus (“AAV”) vectors are also used to transduce cells with target nucleic acids, e.g., in the in vitro production of nucleic acids and peptides, and for in vivo and ex vivo gene therapy procedures (see, e.g., West et al., Virology 160:38-47 (1987); U.S. Pat. No. 4,797,368; WO 93/24641; Kotin, Human Gene Therapy 5:793-801 (1994); Muzyczka, J. Clin. Invest. 94:1351 (1994). Construction of recombinant AAV vectors are described in a number of publications, including U.S. Pat. No. 5,173,414; Tratschin et al., Mol. Cell. Biol. 5:3251-3260 (1985); Tratschin, et al., Mol. Cell. Biol. 4:2072-2081 (1984); Hermonat & Muzyczka, PNAS 81:6466-6470 (1984); and Samulski et al., J. Virol. 63:03822-3828 (1989).

Replication-deficient recombinant adenoviral vectors (Ad) can be produced at high titer and readily infect a number of different cell types. Most adenovirus vectors are engineered such that a transgene replaces the Ad E1a, E1b, and/or E3 genes; subsequently the replication defective vector is propagated in human 293 cells that supply deleted gene function in trans. Ad vectors can transduce multiple types of tissues in vivo, including nondividing, differentiated cells such as those found in liver, kidney and muscle. Conventional Ad vectors have a large carrying capacity. An example of the use of an Ad vector in a clinical trial involved polynucleotide therapy for antitumor immunization with intramuscular injection (Sterman et al., Hum. Gene Ther. 7:1083-9 (1998)). Additional examples of the use of adenovirus vectors for gene transfer in clinical trials include Rosenecker et al., Infection 24:1 5-10 (1996); Sterman et al., Hum. Gene Ther. 9:7 1083-1089 (1998); Welsh et al., Hum. Gene Ther. 2:205-18 (1995); Alvarez et al., Hum. Gene Ther. 5:597-613 (1997); Topf et al., Gene Ther. 5:507-513 (1998); Sterman et al., Hum. Gene Ther. 7:1083-1089 (1998).

Packaging cells are used to form virus particles that are capable of infecting a host cell. Such cells include 293 cells, which package adenovirus, and .psi.2 cells or PA317 cells, which package retrovirus. Viral vectors used in gene therapy are usually generated by a producer cell line that packages a nucleic acid vector into a viral particle. The vectors typically contain the minimal viral sequences required for packaging and subsequent integration into a host (if applicable), other viral sequences being replaced by an expression cassette encoding the protein to be expressed. The missing viral functions are supplied in trans by the packaging cell line. For example, AAV vectors used in gene therapy typically only possess inverted terminal repeat (ITR) sequences from the AAV genome which are required for packaging and integration into the host genome. Viral DNA is packaged in a cell line, which contains a helper plasmid encoding the other AAV genes, namely rep and cap, but lacking ITR sequences. The cell line is also infected with adenovirus as a helper. The helper virus promotes replication of the AAV vector and expression of AAV genes from the helper plasmid. The helper plasmid is not packaged in significant amounts due to a lack of ITR sequences. Contamination with adenovirus can be reduced by, e.g., heat treatment to which adenovirus is more sensitive than AAV.

In many gene therapy applications, it is desirable that the gene therapy vector be delivered with a high degree of specificity to a particular tissue type. Accordingly, a viral vector can be modified to have specificity for a given cell type by expressing a ligand as a fusion protein with a viral coat protein on the outer surface of the virus. The ligand is chosen to have affinity for a receptor known to be present on the cell type of interest. For example, Han et al., Proc. Natl. Acad. Sci. USA 92:9747-9751 (1995), reported that Moloney murine leukemia virus can be modified to express human heregulin fused to gp70, and the recombinant virus infects certain human breast cancer cells expressing human epidermal growth factor receptor. This principle can be extended to other virus-target cell pairs, in which the target cell expresses a receptor and the virus expresses a fusion protein comprising a ligand for the cell-surface receptor. For example, filamentous phage can be engineered to display antibody fragments (e.g., FAB or Fv) having specific binding affinity for virtually any chosen cellular receptor. Although the above description applies primarily to viral vectors, the same principles can be applied to nonviral vectors. Such vectors can be engineered to contain specific uptake sequences which favor uptake by specific target cells.

Gene therapy vectors can be delivered in vivo by administration to an individual patient, typically by systemic administration (e.g., intravenous, intraperitoneal, intramuscular, subdermal, or intracranial infusion) or topical application, as described below. Alternatively, vectors can be delivered to cells ex vivo, such as cells explanted from an individual patient (e.g., lymphocytes, bone marrow aspirates, tissue biopsy) or universal donor hematopoietic stem cells, followed by reimplantation of the cells into a patient, usually after selection for cells which have incorporated the vector.

Ex vivo cell transfection for diagnostics, research, or for gene therapy (e.g., via re-infusion of the transfected cells into the host organism) is well known to those of skill in the art. In a preferred embodiment, cells are isolated from the subject organism, transfected with a ZFP nucleic acid (gene or cDNA), and re-infused back into the subject organism (e.g., patient). Various cell types suitable for ex vivo transfection are well known to those of skill in the art (see, e.g., Freshney et al., Culture of Animal Cells, A Manual of Basic Technique (3rd ed. 1994)) and the references cited therein for a discussion of how to isolate and culture cells from patients).

In one embodiment, stem cells are used in ex vivo procedures for cell transfection and gene therapy. The advantage to using stem cells is that they can be differentiated into other cell types in vitro, or can be introduced into a mammal (such as the donor of the cells) where they will engraft in the bone marrow. Methods for differentiating CD34+ cells in vitro into clinically important immune cell types using cytokines such a GM-CSF, IFN-γ and TNF-α are known (see Inaba et al., J. Exp. Med. 176:1693-1702 (1992)).

Stem cells are isolated for transduction and differentiation using known methods. For example, stem cells are isolated from bone marrow cells by panning the bone marrow cells with antibodies which bind unwanted cells, such as CD4+ and CD8+(T cells), CD45+(panB cells), GR-1 (granulocytes), and lad (differentiated antigen presenting cells) (see Inaba et al., J. Exp. Med. 176:1693-1702 (1992)).

Vectors (e.g., retroviruses, adenoviruses, liposomes, etc.) containing therapeutic TAL-cleavage domain fusion protein nucleic acids can also be administered directly to an organism for transduction of cells in vivo. Alternatively, naked DNA can be administered. Administration is by any of the routes normally used for introducing a molecule into ultimate contact with blood or tissue cells including, but not limited to, injection, infusion, topical application and electroporation. Suitable methods of administering such nucleic acids are available and well known to those of skill in the art, and, although more than one route can be used to administer a particular composition, a particular route can often provide a more immediate and more effective reaction than another route.

Pharmaceutically acceptable carriers are determined in part by the particular composition being administered, as well as by the particular method used to administer the composition. Accordingly, there is a wide variety of suitable formulations of pharmaceutical compositions available, as described below (see, e.g., Remington's Pharmaceutical Sciences, 17th ed., 1989).

With further respect to plants, the polynucleotides and vectors described herein can be used to transform a number of monocotyledonous and dicotyledonous plants and plant cell systems, including dicots such as safflower, alfalfa, soybean, coffee, amaranth, rapeseed (high erucic acid and canola), peanut or sunflower, as well as monocots such as oil palm, sugarcane, banana, sudangrass, corn, wheat, rye, barley, oat, rice, millet, or sorghum. Also suitable are gymnosperms such as fir and pine.

Thus, the methods described herein can be utilized with dicotyledonous plants belonging, for example, to the orders Magniolales, Illiciales, Laurales, Piperales, Aristochiales, Nymphaeales, Ranunculales, Papeverales, Sarraceniaceae, Trochodendrales, Hamamelidales, Eucomiales, Leitneriales, Myricales, Fagales, Casuarinales, Caryophyllales, Batales, Polygonales, Plumbaginales, Dilleniales, Theales, Malvales, Urticales, Lecythidales, Violales, Salicales, Capparales, Ericales, Diapensales, Ebenales, Primulales, Rosales, Fabales, Podostemales, Haloragales, Myrtales, Cornales, Proteales, San tales, Rafflesiales, Celastrales, Euphorbiales, Rhamnales, Sapindales, Juglandales, Geraniales, Polygalales, Umbellales, Gentianales, Polemoniales, Lamiales, Plantaginales, Scrophulariales, Campanulales, Rubiales, Dipsacales, and Asterales. The methods described herein also can be utilized with monocotyledonous plants such as those belonging to the orders Alismatales, Hydrocharitales, Najadales, Triuridales, Commelinales, Eriocaulales, Restionales, Poales, Juncales, Cyperales, Typhales, Bromeliales, Zingiberales, Arecales, Cyclanthales, Pandanales, Arales, Lilliales, and Orchid ales, or with plants belonging to Gymnospermae, e.g., Pinales, Ginkgoales, Cycadales and Gnetales.

The methods can be used over a broad range of plant species, including species from the dicot genera Atropa, Alseodaphne, Anacardium, Arachis, Beilschmiedia, Brassica, Carthamus, Cocculus, Croton, Cucumis, Citrus, Citrullus, Capsicum, Catharanthus, Cocos, Coffea, Cucurbita, Daucus, Duguetia, Eschscholzia, Ficus, Fragaria, Glaucium, Glycine, Gossypium, Helianthus, Hevea, Hyoscyamus, Lactuca, Landolphia, Linum, Litsea, Lycopersicon, Lupinus, Manihot, Majorana, Malus, Medicago, Nicotiana, Olea, Parthenium, Papaver, Persea, Phaseolus, Pistacia, Pisum, Pyrus, Prunus, Raphanus, Ricinus, Senecio, Sinomenium, Stephania, Sinapis, Solanum, Theobroma, Trifolium, Trigonella, Vicia, Vinca, Vilis, and Vigna; the monocot genera Allium, Andropogon, Aragrostis, Asparagus, Avena, Cynodon, Elaeis, Festuca, Festulolium, Heterocallis, Hordeum, Lemna, Lolium, Musa, Oryza, Panicum, Pannesetum, Phleum, Poa, Secale, Sorghum, Triticum, and Zea; or the gymnosperm genera Abies, Cunninghamia, Picea, Pinus, and Pseudotsuga.

A transformed cell, callus, tissue, or plant can be identified and isolated by selecting or screening the engineered cells for particular traits or activities, e.g., those encoded by marker genes or antibiotic resistance genes. Such screening and selection methodologies are well known to those having ordinary skill in the art. In addition, physical and biochemical methods can be used to identify transformants. These include Southern analysis or PCR amplification for detection of a polynucleotide; Northern blots, S1 RNase protection, primer-extension, or RT-PCR amplification for detecting RNA transcripts; enzymatic assays for detecting enzyme or ribozyme activity of polypeptides and polynucleotides; and protein gel electrophoresis, Western blots, immunoprecipitation, and enzyme-linked immunoassays to detect polypeptides. Other techniques such as in situ hybridization, enzyme staining, and immunostaining also can be used to detect the presence or expression of polypeptides and/or polynucleotides. Methods for performing all of the referenced techniques are well known. Polynucleotides that are stably incorporated into plant cells can be introduced into other plants using, for example, standard breeding techniques.

DNA constructs may be introduced into the genome of a desired plant host by a variety of conventional techniques. For reviews of such techniques see, for example, Weissbach & Weissbach Methods for Plant Molecular Biology (1988, Academic Press, N.Y.) Section VIII, pp. 421-463; and Grierson & Corey, Plant Molecular Biology (1988, 2d Ed.), Blackie, London, Ch. 7-9. For example, the DNA construct may be introduced directly into the genomic DNA of the plant cell using techniques such as electroporation and microinjection of plant cell protoplasts, or the DNA constructs can be introduced directly to plant tissue using biolistic methods, such as DNA particle bombardment (see, e.g., Klein et al (1987) Nature 327:70-73). Alternatively, the DNA constructs may be combined with suitable T-DNA flanking regions and introduced into a conventional Agrobacterium tumefaciens host vector. Agrobacterium tumefaciens-mediated transformation techniques, including disarming and use of binary vectors, are well described in the scientific literature. See, for example Horsch et al (1984) Science 233:496-498, and Fraley et al (1983) Proc. Nat'l. Acad. Sci. USA 80:4803. The virulence functions of the Agrobacterium tumefaciens host will direct the insertion of the construct and adjacent marker into the plant cell DNA when the cell is infected by the bacteria using binary T DNA vector (Bevan (1984) Nuc. Acid Res. 12:8711-8721) or the co-cultivation procedure (Horsch et al (1985) Science 227:1229-1231). Generally, the Agrobacterium transformation system is used to engineer dicotyledonous plants (Bevan et al (1982) Ann. Rev. Genet 16:357-384; Rogers et al (1986) Methods Enzymol. 118:627-641). The Agrobacterium transformation system may also be used to transform, as well as transfer, DNA to monocotyledonous plants and plant cells. See Hernalsteen et al (1984) EMBO J 3:3039-3041; Hooykass-Van Slogteren et al (1984) Nature 311:763-764; Grimsley et al (1987) Nature 325:1677-179; Boulton et al (1989) Plant Mol. Biol. 12:31-40; and Gould et al (1991) Plant Physiol. 95:426-434.

Alternative gene transfer and transformation methods include, but are not limited to, protoplast transformation through calcium-, polyethylene glycol (PEG)- or electroporation-mediated uptake of naked DNA (see Paszkowski et al. (1984) EMBO J3:2717-2722, Potrykus et al. (1985) Molec. Gen. Genet. 199:169-177; Fromm et al. (1985) Proc. Nat. Acad. Sci. USA 82:5824-5828; and Shimamoto (1989) Nature 338:274-276) and electroporation of plant tissues (D'Halluin et al. (1992) Plant Cell 4:1495-1505). Additional methods for plant cell transformation include microinjection, silicon carbide mediated DNA uptake (Kaeppler et al. (1990) Plant Cell Reporter 9:415-418), and microprojectile bombardment (see Klein et al. (1988) Proc. Nat. Acad. Sci. USA 85:4305-4309; and Gordon-Kamm et al. (1990) Plant Cell 2:603-618).

The disclosed methods and compositions can be used to insert exogenous sequences into a predetermined location in a plant cell genome. This is useful inasmuch as expression of an introduced transgene into a plant genome depends critically on its integration site. Accordingly, genes encoding, e.g., nutrients, antibiotics or therapeutic molecules can be inserted, by targeted recombination, into regions of a plant genome favorable to their expression.

Transformed plant cells which are produced by any of the above transformation techniques can be cultured to regenerate a whole plant which possesses the transformed genotype and thus the desired phenotype. Such regeneration techniques rely on manipulation of certain phytohormones in a tissue culture growth medium, typically relying on a biocide and/or herbicide marker which has been introduced together with the desired nucleotide sequences. Plant regeneration from cultured protoplasts is described in Evans, et al., “Protoplasts Isolation and Culture” in Handbook of Plant Cell Culture, pp. 124-176, Macmillian Publishing Company, New York, 1983; and Binding, Regeneration of Plants, Plant Protoplasts, pp. 21-73, CRC Press, Boca Raton, 1985. Regeneration can also be obtained from plant callus, explants, organs, pollens, embryos or parts thereof. Such regeneration techniques are described generally in Klee et al (1987) Ann. Rev. of Plant Phys. 38:467-486.

Nucleic acids introduced into a plant cell can be used to confer desired traits on essentially any plant. A wide variety of plants and plant cell systems may be engineered for the desired physiological and agronomic characteristics described herein using the nucleic acid constructs of the present disclosure and the various transformation methods mentioned above. In preferred embodiments, target plants and plant cells for engineering include, but are not limited to, those monocotyledonous and dicotyledonous plants, such as crops including grain crops (e.g., wheat, maize, rice, millet, barley), fruit crops (e.g., tomato, apple, pear, strawberry, orange), forage crops (e.g., alfalfa), root vegetable crops (e.g., carrot, potato, sugar beets, yam), leafy vegetable crops (e.g., lettuce, spinach); flowering plants (e.g., petunia, rose, chrysanthemum), conifers and pine trees (e.g., pine fir, spruce); plants used in phytoremediation (e.g., heavy metal accumulating plants); oil crops (e.g., sunflower, rape seed) and plants used for experimental purposes (e.g., Arabidopsis).

Thus, the disclosed methods and compositions have use over a broad range of plants, including, but not limited to, species from the genera Asparagus, Avena, Brassica, Citrus, Citrullus, Capsicum, Cucurbita, Daucus, Glycine, Hordeum, Lactuca, Lycopersicon, Malus, Manihot, Nicotiana, Oryza, Persea, Pisum, Pyrus, Prunus, Raphanus, Secale, Solanum, Sorghum, Triticum, Vitis, Vigna, and Zea. One of skill in the art will recognize that after the expression cassette is stably incorporated in transgenic plants and confirmed to be operable, it can be introduced into other plants by sexual crossing. Any of a number of standard breeding techniques can be used, depending upon the species to be crossed.

A transformed plant cell, callus, tissue or plant may be identified and isolated by selecting or screening the engineered plant material for traits encoded by the marker genes present on the transforming DNA. For instance, selection may be performed by growing the engineered plant material on media containing an inhibitory amount of the antibiotic or herbicide to which the transforming gene construct confers resistance. Further, transformed plants and plant cells may also be identified by screening for the activities of any visible marker genes (e.g., the β-glucuronidase, luciferase, B or C1 genes) that may be present on the recombinant nucleic acid constructs. Such selection and screening methodologies are well known to those skilled in the art.

Physical and biochemical methods also may be used to identify plant or plant cell transformants containing inserted gene constructs. These methods include but are not limited to: 1) Southern analysis or PCR amplification for detecting and determining the structure of the recombinant DNA insert; 2) Northern blot, S1 RNase protection, primer-extension or reverse transcriptase-PCR amplification for detecting and examining RNA transcripts of the gene constructs; 3) enzymatic assays for detecting enzyme or ribozyme activity, where such gene products are encoded by the gene construct; 4) protein gel electrophoresis, Western blot techniques, immunoprecipitation, or enzyme-linked immunoassays, where the gene construct products are proteins. Additional techniques, such as in situ hybridization, enzyme staining, and immunostaining, also may be used to detect the presence or expression of the recombinant construct in specific plant organs and tissues. The methods for doing all these assays are well known to those skilled in the art.

Effects of gene manipulation using the methods disclosed herein can be observed by, for example, northern blots of the RNA (e.g., mRNA) isolated from the tissues of interest. Typically, if the amount of mRNA has increased, it can be assumed that the corresponding endogenous gene is being expressed at a greater rate than before. Other methods of measuring gene and/or CYP74B activity can be used. Different types of enzymatic assays can be used, depending on the substrate used and the method of detecting the increase or decrease of a reaction product or by-product. In addition, the levels of and/or CYP74B protein expressed can be measured immunochemically, i.e., ELISA, RIA, EIA and other antibody based assays well known to those of skill in the art, such as by electrophoretic detection assays (either with staining or western blotting). The transgene may be selectively expressed in some tissues of the plant or at some developmental stages, or the transgene may be expressed in substantially all plant tissues, substantially along its entire life cycle. However, any combinatorial expression mode is also applicable.

The present disclosure also encompasses seeds of the transgenic plants described above wherein the seed has the transgene or gene construct. The present disclosure further encompasses the progeny, clones, cell lines or cells of the transgenic plants described above wherein said progeny, clone, cell line or cell has the transgene or gene construct.

### Delivery Vehicles

An important factor in the administration of polypeptide compounds, such as a TAL-cleavage or ZFN-cleavage domain fusion protein, is ensuring that the polypeptide has the ability to traverse the plasma membrane of a cell, or the membrane of an intracellular compartment such as the nucleus. Cellular membranes are composed of lipid-protein bilayers that are freely permeable to small, nonionic lipophilic compounds and are inherently impermeable to polar compounds, macromolecules, and therapeutic or diagnostic agents. However, proteins and other compounds such as liposomes have been described, which have the ability to translocate polypeptides such as TAL-cleavage domain fusion proteins across a cell membrane.

For example, “membrane translocation polypeptides” have amphiphilic or hydrophobic amino acid subsequences that have the ability to act as membrane-translocating carriers. In one embodiment, homeodomain proteins have the ability to translocate across cell membranes. The shortest internalizable peptide of a homeodomain protein, Antennapedia, was found to be the third helix of the protein, from amino acid position 43 to 58 (see, e.g., Prochiantz, Current Opinion in Neurobiology 6:629-634 (1996)). Another subsequence, the h (hydrophobic) domain of signal peptides, was found to have similar cell membrane translocation characteristics (see, e.g., Lin et al., J. Biol. Chem. 270:14255-14258 (1995)).

Examples of peptide sequences which can be linked to a protein, for facilitating uptake of the protein into cells, include, but are not limited to: an 11 amino acid peptide of the tat protein of HIV; a 20 residue peptide sequence which corresponds to amino acids 84-103 of the p16 protein (see Fahraeus et al., Current Biology 6:84 (1996)); the third helix of the 60-amino acid long homeodomain of Antennapedia (Derossi et al., J. Biol. Chem. 269:10444 (1994)); the h region of a signal peptide such as the Kaposi fibroblast growth factor (K-FGF) h region (Lin et al., supra); or the VP22 translocation domain from HSV (Elliot & O'Hare, Cell 88:223-233 (1997)). Other suitable chemical moieties that provide enhanced cellular uptake may also be chemically linked to ZFPs. Membrane translocation domains (i.e., internalization domains) can also be selected from libraries of randomized peptide sequences. See, for example, Yeh et al. (2003) Molecular Therapy 7(5):S461, Abstract #1191.

Toxin molecules also have the ability to transport polypeptides across cell membranes. Often, such molecules (called “binary toxins”) are composed of at least two parts: a translocation/binding domain or polypeptide and a separate toxin domain or polypeptide. Typically, the translocation domain or polypeptide binds to a cellular receptor, and then the toxin is transported into the cell. Several bacterial toxins, including Clostridium perfringens iota toxin, diphtheria toxin (DT), Pseudomonas exotoxin A (PE), pertussis toxin (PT), Bacillus anthracis toxin, and pertussis adenylate cyclase (CYA), have been used to deliver peptides to the cell cytosol as internal or amino-terminal fusions (Arora et al., J. Biol. Chem., 268:3334-3341 (1993); Perelle et al., Infect. Immun., 61:5147-5156 (1993); Stennark et al. J. Cell Biol. 113:1025-1032 (1991); Donnelly et al., PNAS 90:3530-3534 (1993); Carbonetti et al., Abstr. Annu. Meet. Am. Soc. Microbiol. 95:295 (1995); Sebo et al. Infect. Immun. 63:3851-3857 (1995); Klimpel et al. PNAS U.S.A. 89:10277-10281 (1992); and Novak et al., J. Biol. Chem. 267:17186-17193 1992)).

Such peptide sequences can be used to translocate TAL-cleavage or ZFN-cleavage domain fusion proteins across a cell membrane. TAL-cleavage or ZFN-cleavage domain fusion proteins can be conveniently fused to or derivatized with such sequences. Typically, the translocation sequence is provided as part of a fusion protein. Optionally, a linker can be used to link the TAL-cleavage or ZFN-cleavage domain fusion protein and the translocation sequence. Any suitable linker can be used, e.g., a peptide linker.

The TAL-cleavage or ZFN-cleavage domain fusion protein can also be introduced into an animal cell, preferably a mammalian cell, via a liposomes and liposome derivatives such as immunoliposomes. The term “liposome” refers to vesicles comprised of one or more concentrically ordered lipid bilayers, which encapsulate an aqueous phase. The aqueous phase typically contains the compound to be delivered to the cell,

The liposome fuses with the plasma membrane, thereby releasing the drug into the cytosol. Alternatively, the liposome is phagocytosed or taken up by the cell in a transport vesicle. Once in the endosome or phagosome, the liposome either degrades or fuses with the membrane of the transport vesicle and releases its contents.

In current methods of drug delivery via liposomes, the liposome ultimately becomes permeable and releases the encapsulated compound (in this case, a TAL-cleavage or ZFN-cleavage domain fusion protein) at the target tissue or cell. For systemic or tissue specific delivery, this can be accomplished, for example, in a passive manner wherein the liposome bilayer degrades over time through the action of various agents in the body. Alternatively, active drug release involves using an agent to induce a permeability change in the liposome vesicle. Liposome membranes can be constructed so that they become destabilized when the environment becomes acidic near the liposome membrane (see, e.g., PNAS 84:7851 (1987); Biochemistry 28:908 (1989)). When liposomes are endocytosed by a target cell, for example, they become destabilized and release their contents. This destabilization is termed fusogenesis. Dioleoylphosphatidylethanolamine (DOPE) is the basis of many “fusogenic” systems.

The disclosed methods for targeted recombination can be used to replace any genomic sequence with a homologous, non-identical sequence. For example, a mutant genomic sequence can be replaced by its wild-type counterpart, thereby providing methods for treatment of e.g., genetic disease, inherited disorders, cancer, and autoimmune disease. In like fashion, one allele of a gene can be replaced by a different allele using the methods of targeted recombination disclosed herein. Exemplary genetic diseases include, but are not limited to, achondroplasia, achromatopsia, acid maltase deficiency, adenosine deaminase deficiency (OMIM No. 102700), adrenoleukodystrophy, aicardi syndrome, alpha-1 antitrypsin deficiency, alpha-thalassemia, androgen insensitivity syndrome, apert syndrome, arrhythmogenic right ventricular, dysplasia, ataxia telangictasia, barth syndrome, beta-thalassemia, blue rubber bleb nevus syndrome, canavan disease, chronic granulomatous diseases (CGD), cri du chat syndrome, cystic fibrosis, dercum's disease, ectodermal dysplasia, fanconi anemia, fibrodysplasia ossificans progressive, fragile X syndrome, galactosemis, Gaucher's disease, generalized gangliosidoses (e.g., GM 1), hemochromatosis, the hemoglobin C mutation in the 6.sup.th codon of beta-globin (HbC), hemophilia, Huntington's disease, Hurler Syndrome, hypophosphatasia, Kinefleter syndrome, Krabbes Disease, Langer-Giedion Syndrome, leukocyte adhesion deficiency (LAD, OMIM No. 116920), leukodystrophy, long QT syndrome, Marfan syndrome, Moebius syndrome, mucopolysaccharidosis (MPS), nail patella syndrome, nephrogenic diabetes insipdius, neurofibromatosis, Neimann-Pick disease, osteogenesis imperfecta, porphyria, Prader-Willi syndrome, progeria, Proteus syndrome, retinoblastoma, Rett syndrome, Rubinstein-Taybi syndrome, Sanfilippo syndrome, severe combined immunodeficiency (SCID), Shwachman syndrome, sickle cell disease (sickle cell anemia), Smith-Magenis syndrome, Stickler syndrome, Tay-Sachs disease, Thrombocytopenia Absent Radius (TAR) syndrome, Treacher Collins syndrome, trisomy, tuberous sclerosis, Turner's syndrome, urea cycle disorder, von Hippel-Landau disease, Waardenburg syndrome, Williams syndrome, Wilson's disease, Wiskott-Aldrich syndrome, X-linked lymphoproliferative syndrome (XLP, OMIM No. 308240).

Additional exemplary diseases that can be treated by targeted DNA cleavage and/or homologous recombination include acquired immunodeficiencies, lysosomal storage diseases (e.g., Gaucher's disease, GM 1, Fabry disease and Tay-Sachs disease), mucopolysaccahidosis (e.g. Hunter's disease, Hurler's disease), hemoglobinopathies (e.g., sickle cell diseases, HbC, α-thalassemia, 3-thalassemia) and hemophilias.

In certain cases, alteration of a genomic sequence in a pluripotent cell (e.g., a hematopoietic stem cell) is desired. Methods for mobilization, enrichment and culture of hematopoietic stem cells are known in the art. See for example, U.S. Pat. Nos. 5,061,620; 5,681,559; 6,335,195; 6,645,489 and 6,667,064. Treated stem cells can be returned to a patient for treatment of various diseases including, but not limited to, SCID and sickle-cell anemia.

In many of these cases, a region of interest comprises a mutation, and the donor polynucleotide comprises the corresponding wild-type sequence. Similarly, a wild-type genomic sequence can be replaced by a mutant sequence, if such is desirable. For example, overexpression of an oncogene can be reversed either by mutating the gene or by replacing its control sequences with sequences that support a lower, non-pathologic level of expression. As another example, the wild-type allele of the ApoAI gene can be replaced by the ApoAI Milano allele, to treat atherosclerosis. Indeed, any pathology dependent upon a particular genomic sequence, in any fashion, can be corrected or alleviated using the methods and compositions disclosed herein.

Targeted cleavage and targeted recombination can also be used to alter non-coding sequences (e.g., regulatory sequences such as promoters, enhancers, initiators, terminators, splice sites) to alter the levels of expression of a gene product. Such methods can be used, for example, for therapeutic purposes, functional genomics and/or target validation studies.

### Example 1

### Monomer Architecture of TAL Nuclease for Efficient In Vivo Gene Inactivation

Artificial endonucleases hold great promise in basic and applied research, even in therapeutic treatment of genetic disorders. The improving zinc finger nucleases (ZFN) and the emerging TAL effector nucleases (TALEN) are the most prominent in the field. These two types of artificial nucleases are comprised of DNA binding domains (TAL effectors or zinc finger proteins) each linked to the nuclease cleavage domain of FokI restriction enzyme. The DNA binding domains of these two kinds can be designed and manipulated to recognize the user chosen genomic sites in living cells and direct the linked nuclease domains there to cleave the two DNA strands. The nature of FokI nuclease domain requires self-dimerization for cutting DNA such that two engineered nucleases (a dimer) must be present at the specific site with one nuclease binding to one strand and the other to an adjacent site of the opposite strand. The two binding sites must be appropriately separated so the two FokI cleavage domains (one from each engineered nuclease) can dimerize and cause double strand break (DSB). There are a number of limitations to its applicability for this kind of architecture. For example, two nucleases have to be made for each target site of interest; the overall cleaving efficiency will depend on the coordination of these two nucleases; requirement of two sites and appropriate length of spacer collectively limit the choice of potential target sites for engineering suitable nucleases. Therefore, it is desirable to develop an architecture that enables single engineered nucleases to be functional as efficiently as dimeric nucleases, here in case of TALENs and ZFNs. Here, we discover that the DNA cleavage domain of homing nuclease I-TevI is amenable to fusion with TAL effector proteins and zinc finger proteins. The fusion proteins exhibited the DNA binding specificity of ZF and TALEs and DNA cleaving activity to double strands near the binding sites as monomer. Both monomeric ZFNs and TALENs when expressed in yeast cells were able to induce DSBs in the plasmid carrying the respective target sequences and stimulate the homologous recombination of two duplicated regions in the yeast single strand annealing (SSA) assay. When expressed in year cells, the monomeric nucleases induced gene disruption at the expected sites where single stretches of the target DNA sequences were present. Taken together, the I-TevI DNA cleavage domain and the TALEs and ZFPs can be linked to form active monomeric nucleases capable of modifying genes at specific sites.

### Results:

I. Structure, DNA and Amino Acid Sequences of Monomer TAL Nucleases

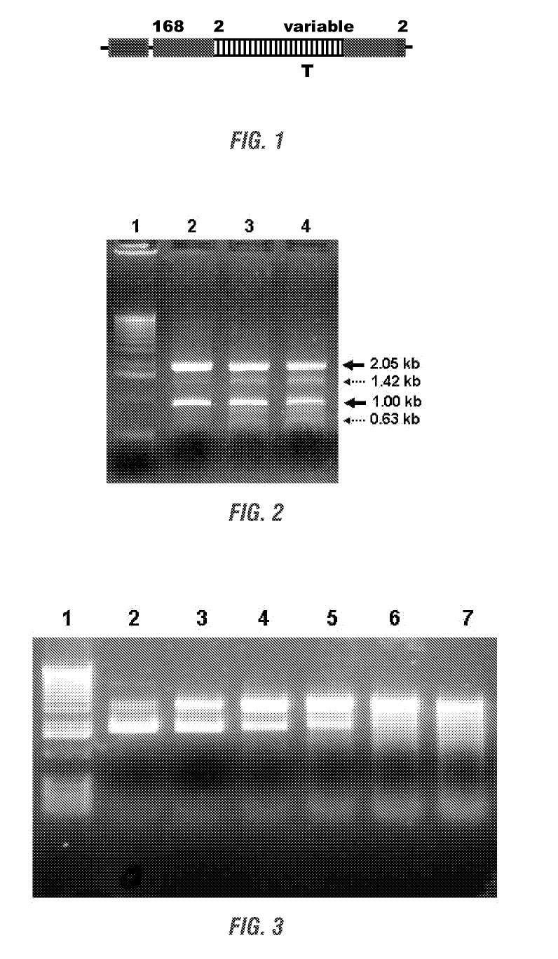

The structure of our monomer TAL and zinc finger nucleases is the DNA cleavage domain of the homing endonuclease I-TevI (referred to as Tv domain) fused with the TAL effectors (naturally occurring and custom-made) and zinc finger proteins (e.g. Zif268 in this study). Tv is located at the N-terminus while TAL effector (full length or truncated) and zinc finger proteins at the C-terminus of the hybrid proteins (FIG. 1).

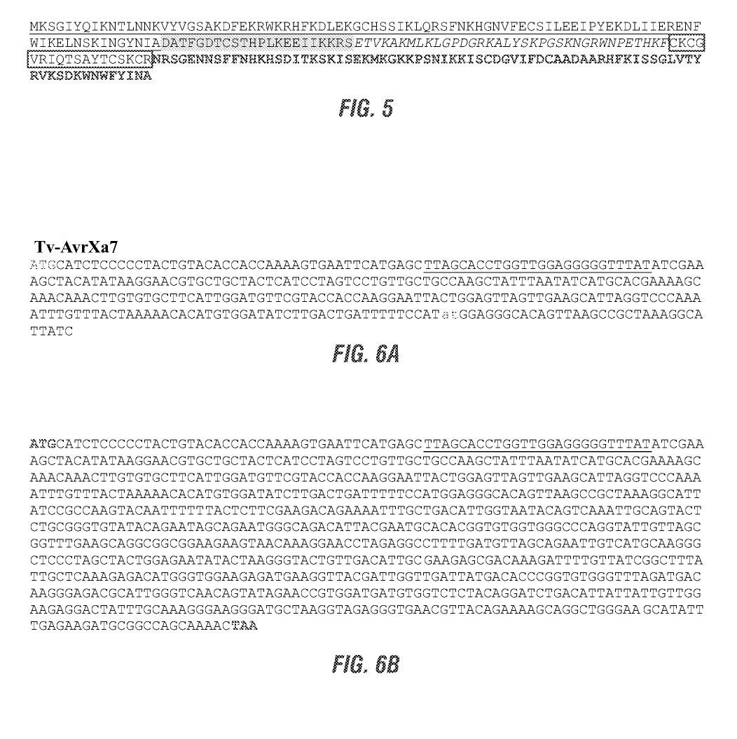

Amino acid sequence of I-TevI homing endonuclease, GenBank accession number, AAD42521 SEQ ID NO: 1 and FIG. 5.
Amino acids 1-92 (underlined) is the catalytic GIY-YIG domain (SEQ ID NO: 13); 93-114 as (shaded in grey) is the deletion intolerant domain (SEQ ID NO: 14); 115-149 as (italic) is the deletion tolerant domain (SEQ ID NO: 15); 150-168 as (shade in yellow) is the zinc finger domain (SEQ ID NO: 16); 169-254 as (in red) is the DNA binding domain (SEQ ID NO:7
N-terminus of 168 amino acids (Tv) is used to fuse with either TAL effectors or zinc finger proteins for monomer TAL nucleases or monomer zinc finger nucleases (FIG. 1)(SEQ ID NO:2, FIG. 5(b). SEQ ID NO:3 is the DNA sequence encoding the Tv domain.
Primers used for PCR amplification of Tv-coding DNA fragment are forward primer 5′-(SEQ ID NO;4) and reverse primer 5′-SEQ ID NO:5). The template of PCR is T4 phage DNA.

FIG. 1. Structure of monomer TAL nuclease.

Full-length TAL effector (TALE) (e.g., AvrXa7) is fused at the C-terminus of endonuclease Tev-I (Tv) (N-terminal 168 amino acid DNA cleavage domain of Tev-I (SEQ ID NO:2). The Tv is linked through restriction site BamHI with TAL effector proteins which contain the varying number of repeats. The DNA coding sequence of Tv-AvrXa7 is SEQ ID NO:6.

The Tv-AvrXa7 EBE (effector binding element) sequence is shown in SEQ ID NO:7:

The DNA coding sequence of Tv-U3a-R IS SEQ ID NO:8) (U3a-R is a custom-made TAL effector for targeting yeast URA3 gene.)

The Tv-U3a-R EBE sequence is SEQ ID NO:9:

The Tv-U3b-R coding sequence is SEQ ID NO: 10 (U3b-R is another custom-made TAL effector targeting another site within the yeast URA3 gene.)

The Tv-U3b-R EBE sequence is SEQ ID NO: 11

The Tv-Zif268 coding sequence is SEQ ID NO: 12 and the

Tv-Zif268 EBE sequence is GCGTGGGC

### Example 2

### Monomer TAL Nuclease Tv-AvrXa7 In Vitro Activity

The chimeric gene of Tv-AvrXa7 was cloned into pPROEX HTb (Invitrogen, Carlsbad, Calif., USA) by ligating the Bg1II-HindIII fragment of Tv-AvrXa7 into BamHI and HindIII digested vector for expression and purification of recombinant protein in bacterial expression system (E. col). The recombinant protein was purified with the 6 histidine tag based Ni-NTA agarose (Qiagen, Valencia, Calif., USA) and the protein concentrations were determined using the BioRad Bradford protein quantification kit (BioRad, Hercules, Calif., USA). For in vitro DNA digestion of Tv-AvrXa7, a 406 bp genomic region of the rice Os11N3 gene encompassing the AvrXa7 EBE was PCR amplified and cloned into pTOPO cloning vector, resulting in plasmid DNA pTOPO/11N3. The buffer condition for in vitro digestion is Tris-HCl (15 mM, pH 7.5), KCl (40 mM), DTT (1 mM), glycerol (2%), poly(dI-dC) (50 ng/ul), EDTA (0.2 mM) and the Tv-AvrXa7 concentrations are indicated in each specific treatment.

FIG. 2. TevI-AvrXa7 Nuclease Activities on Plasmid DNA.

Agarose gel image of plasmid DNA pTOPO/11N3 (containing effector binding element, EBE, of AvrXa7) treated with restriction enzyme MluI and subsequently the purified recombinant Tv-AvrXa7. MluI completely cuts pTOPO/11N3 into two fragments (2.05 kb and 1.0 kb as indicated by the arrows). The 2.05 kb AvrXa7-EBE-containing fragment is cleaved by Tv-AvrXa7 into 1.42 and 0.63 kb as expected (1.42 and 0.63 kb). Lane 1, 1 kb marker, lanes 2, 3, 4 represent the same amount (250 ng) of DNA treated with different concentrations (0.25 ug, 0.5 ug, and 0.75 ug) of Tv-AvrXa7.

FIG. 3. Tv-AvrXa7 Nuclease Activity in Linearizing Plasmid DNA.

A gel image of plasmid pTOPO/11N3 treated with Tv-AvrXa7. Lower band is the super coiled plasmid. The upper band is the linearized plasmid. Lane 1 is 1 kb marker. Lane 2-7 is the plasmid pTOPO/11N3 treated with 0, 0.1, 0.2, 0.3, 0.4, 0.5 ug of TvI-AvrXa7 individually.

III. Monomer TAL Nuclease Activity In Vivo Plasmid-Borne Homologous Recombination

The ability of monomer TAL and zinc finger nucleases to bind and cleave target sequence in vivo was tested by using yeast single-strand annealing (SSA) assay. In this assay, a “reporter” construct (plasmid) is coexpressed with an effector construct in yeast cells. The reporter construct contains a divided LacZ gene in which a duplicated 125 bp segment of the LacZ coding region has been created. The direct repeats are separated by the single sequence of individual AvrXa7 EBE, U3b-R EBE and Zif268 EBE. It is expected that the direct DNA repeats will undergo homologous recombination at high efficiency when a double strand break is created between the repeats by the respective TAL and zinc finger nucleases, resulting in a reconstituted and functional LacZ gene. Measurement of 3-galactosidase (LacZ gene product) enzymatic activity was used to reflect the activity of effector nucleases in the presence of various target sequences. Yeast vector pCP5 was used for construction of reporter plasmids each with EBE of Tv-AvrXa7, Tv-U3b-R, and Tv-Zif268 by using restriction sites of Bg1II and SpeI. The yeast expression vector pCP3 was used to construct the effector plasmids of Tv-AvrXa7, Tv-U3b-R, and Tv-Zif268 by using the restriction sites of BamHI and SpeI. The yeast strain YPH500 was used for the SSA assay of the nucleases on their respective target sequences. Our data clearly demonstrated the ability of monomer TAL and zinc finger nucleases in targeting the specific DNA sequences and stimulating homologous recombination of the plasmid-borne reporter gene.

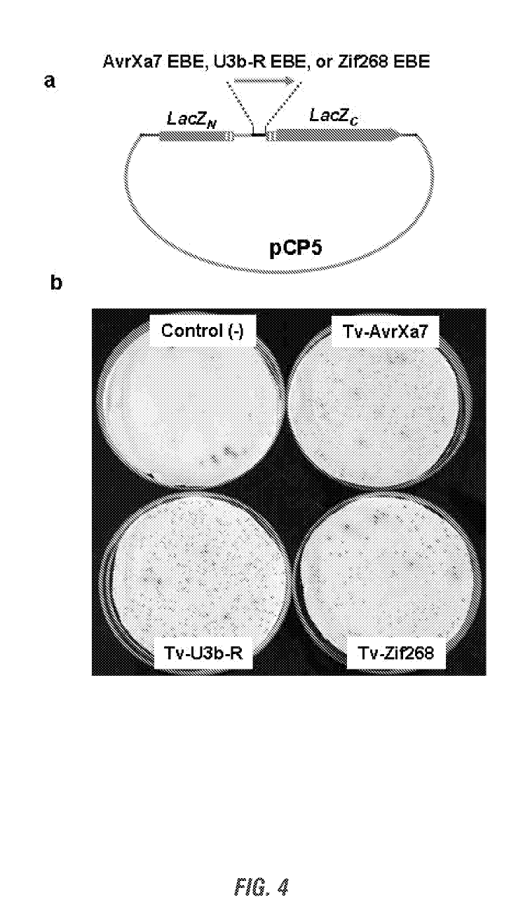

FIG. 4. Homologous Recombination of Plasmid-Borne Reporter Gene in Yeast Mediated by Monomer TALENs. (a)

Constructs of the reporter gene LacZ. Two LacZ fragments (LacZN and LacZC) sharing a duplicated 125 bp portion (hatched boxes) of LacZ coding region were separated by a sequence of the respective TAL effector or zinc finger protein binding sites (AvrXa7 EBE, U3b-R EBE, or Zif268 EBE). (b) Yeast colonies stained with the substrate (X-gal, 5-bromo-4-chloro-3-indolyl-3-d-galactopyranoside) of the LacZ gene product (β-galactosidase). The color density of colonies in blue reflects the activities of monomer TAL nucleases in cleaving and stimulating the homologous recombination of the two duplicated LacZ regions of the reporter constructs in yeast cells. Yeast colonies with effector construct (control, empty vector lacking any nuclease gene; Tv-avrXa7, Tv-U3b-R, and Tv-Zif268) and their respective target sequences were transferred through colony-lift onto filter membrane and stained with X-gal for 2 hrs and photographed.

IV. Monomer TAL Nuclease Induced High Efficient Gene Inactivation in Yeast Cells

We also expanded the experimentation to test the ability of monomer TAL nucleases in targeting the site-specific sequences for gene inactivation in the chromosomal context. Three TAL effectors (naturally occurring AvrXa7 and custom-made U3a-R and U3b-R), all as the DNA binding proteins, were individually fused with the DNA cleavage domain (168 amino acids) of Tev-I (Tv). The three chimeric genes were individually expressed in yeast strain YPH500c containing the respectively effector targeting sites. YPH500c contains the functional URA3 gene with AvrXa7 target DNA sequences integrated into the URA3 coding region downstream of the translational start codon (ATG) (as described in Nucleic Acids Research, 39:6315-6325). The custom-made TAL effectors U3a-R and U3b-R target their respective DNA regions within the coding region of URA3. The transformants were grown on synthetic complete (SC) medium lacking histidine for 5 days before plating on the SC medium containing 0.1% 5-fluoroorotic acid (5-FOA) for selection of resistant colonies and in parallel on SC medium without 5-FOA to test for plating efficiency. Genomic DNA extracted from a number of 5-FOA-resistant colonies for each monomer nuclease was used for PCR amplification of the relevant regions. The PCR products were sequenced using the respective primers. Our data (Table 1) shows the efficient gene inactivation by the monomeric TAL nucleases. URA3 mutants were obtained at a rate of ˜10⁴ to ˜10³ mutants/total cells. In contrast, ˜10⁶ yeast cells carrying the plasmid lacking a functional TAL nuclease gene (Empty vector) yielded a few colonies resistant to 5-FOA. Sequence analysis of PCR-amplified genomic DNA from the relevant target sites in some mutants confirmed the existence of mutagenic insertions within the coding region of URA3 gene.

TABLE 1
Frequency of gene inactivation by
monomer TAL nucleases (Tv-TALE)

| | Tv-AvrXa7 | Tv-U3a-R | Tv-U3b-R | Empty vector |
|---|---|---|---|---|
| URA3 | 5.9 × 10⁻⁴ | 2.36 × 10⁻⁴ | 1.8 × 10⁻³ | <1.4 × 10⁻⁶ |

### Genotyping Sequences of Some 5-FOA Resistant Yeast Colonies:

### 1. Tv-AvrXa7

Four colonies were genotyped and were found to contain the same mutation. See FIG. 6A (SEQ ID NO: 18). The underlined 26 base pairs were the binding site of AvrXa7, the red lower “at” is the inserted sequence which caused the frame shift of URA3 open reading frame (thus, mutagenic and conferring resistance to 5-FOA). See FIG. 6B (SEQ ID NO: 19) for the functional URA3 coding sequence.

### 2. Tv-U3b-R

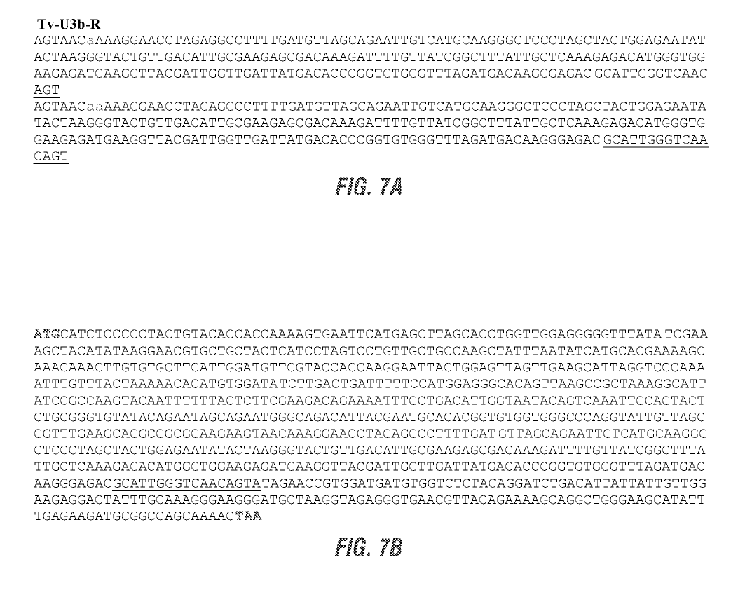

Eight colonies were genotyped and seven were found to contain an “a” insertion and one to contain an “aa” insertion. The mutations are red and lower cases. See FIG. 7A (SEQ ID NO:20).

The functional URA3 coding sequence is shown in FIG. 7B (SEQ ID NO:21)

The contents of any patents, patent applications, and references cited throughout this specification are hereby incorporated by reference in their entireties.

Those skilled in the art will recognize, or be able to ascertain using no more than routine experimentation, many equivalents to the specific embodiments of the invention described herein. Such equivalents are intended to be encompassed by the following claims.

1. A method for modifying cellular DNA in a targeted manner comprising: introducing to said cell a fusion protein comprising (a) a TAL type III effector or zinc finger
binding domain and (b) a homing nuclease cleavage domain from 1-TevI; wherein said fusion protein has a monomer architecture and further so that cellular DNA is cleaved in the region targeted by TAL effector binding domain.

2. The method of claim 1 wherein said I-Tev cleavage domain protein is selected from the group consisting of:
(a) SEQ ID NO:2 or
(b) a polypeptide comprising at least 90% homology to a polypeptide of SEQ ID NO:2, or
(c) conservatively modified variants (a) or (b).

3. The method of claim 1 wherein said I-TevI cleavage domain is SEQ ID NO:2.

4. The method of claim 1 wherein said protein is introduced by incorporation of a heterologous nucleic acid sequence to said cell comprising a sequence which encodes I-TevI endonuclease domain and a binding domain.

5. The method of claim 4 wherein said binding domain is a zinc finger binding domain.

6. The method of claim 4 wherein said binding domain is a TAL effector binding domain.

7. The method of claim 4 wherein said nucleic acid sequence further includes a sequence to be incorporated into said chromatin.

8. The method of claim 4 wherein said I-TevI nucleic acid sequence is selected from the group consisting of
(a) SEQ ID NO:3
(b) a nucleic acid sequence which is at least 90% homology to SEQ ID NO:3, or
(c) which hybridizes to a nucleic acid sequence of SEQ ID NO:3 under conditions of high stringency and
(d) which encodes a protein if SEQ ID NO:2

9. The method of claim 1 wherein said TAL type III effector is AvrXa7.

10. The method of claim 1 wherein said zinc finger protein is Zif268.

11. A genetically modified cell or an ancestor thereof which has been genetically modified by the process of claim 1.

12. A fusion protein comprising a TAL type III effector sequence from Xanthomonas oryzae pv. oryzae or a zinc finger binding sequence and a ITev-I cleavage domain.

13. The fusion protein if claim 12 wherein said ITev-I cleavage domain is selected from the group consisting of (a) SEQ ID NO:2 (b) a polypeptide comprising at least 90% homology to a polypeptide of SEQ ID NO:2, or (c) conservatively modified variants (a) or (b).

14. The fusion protein of claim 12 wherein said zinc finger protein domain is Zif268.

15. The fusion protein of claim 14 wherein said protein is encoded by SEQ ID NO:12

16. The fusion protein of claim 12 wherein said TAL type III effector sequence is AvrXa7.

17. The fusion protein of claim 16 wherein said protein is encoded by SEQ ID NO:6.

18. A genetically modified cell comprising the fusion protein of claim 12.

19. A nucleic acid sequence encoding a I-TevI protein and a TAL effector or zinc finger protein said 1-TevI nucleic acid sequence selected from the group consisting of
(e) SEQ ID NO:3
(f) a nucleic acid sequence which is at least 90% homology to SEQ ID NO:3, or
(g) which hybridizes to a nucleic acid sequence of SEQ ID NO:3 under conditions of high stringency and
(h) which encodes a protein if SEQ ID NO:2.

20. An expression cassette comprising the nucleic acid sequence of claim 14 operably linked to a promoter sequence.

21. A vector comprising the expression cassette of claim 15.

22. A cell comprising the vector of claim 16.

23. A method for targeted recombination in a cell at comprising: introducing to said cell a fusion protein of monomer architecture and comprising a AvrXa7 TAL
type III effector binding domain target sequence and a single I-TevI cleavage domain; so that cellular chromatin is cleaved in the region targeted by TAL effector binding domain so
that homologous recombination may occur.

24. The method of claim 23 wherein said binding domain target sequence is determined according to the following code of 12^{th} and 13^{th} amino acids of the AvrXa7 TAL type III effector binding domain:
HD C/G or A/T
NI A/T or G/C or C/G
NG T/A
NS A/T or T/A or C/G
NN C/G or A/T or G/C
N* C/G or T/A or A/T
HG T/A

25. A method for targeted recombination in a cell at comprising: introducing to said cell a fusion protein of monomer architecture and comprising a zinc protein
binding domain target sequence and a single I-TevI cleavage domain; so that cellular chromatin is cleaved in the region targeted by TAL effector binding domain so
that homologous recombination may occur.

26. The method of claim 25 wherein said zinc finger domain is Zif268.
